(19) United States
(12) Patent Application Publication
(10) Pub. No.: US 2026/0364433 A1
(43) Pub. Date: Nov. 19, 2026

(54) REAL-TIME BODY ALIGNMENT GUIDANCE SYSTEM

(71) Applicant: Snap Inc., Santa Monica, CA (US)

(72) Inventors: Terek Judi, Venice, CA (US); Ihor Kuzin, Los Angeles, CA (US); Artur Sydoran, Playa del Rey, CA (US); Summer Wu, Santa Monica, CA (US)

(21) Appl. No.: 19/677,575

(22) Filed: May 14, 2026

Related U.S. Application Data
(60) Provisional application No. 63/805,421, filed on May 14, 2025.

(51) Int. Cl. A63B 71/06; A63B 24/00; G06T 7/20; G06T 7/70; G06V 40/10; G06V 40/20

(52) U.S. Cl. CPC A63B 71/0622; A63B 24/0006; A63B 24/0075; G06T 7/20; G06T 7/70; G06V 40/10; G06V 40/23; A63B 2024/0012; A63B 2071/0638; A63B 2071/0666; A63B 2220/10; A63B 2220/806; G06T 2207/10016; G06T 2207/30196

(57) ABSTRACT

Examples relate to systems and methods for providing physical activity guidance through augmented reality. A mobile device positioned to capture a user performing physical activities serves as an external body tracker, capturing movement data through its camera. One or more processors analyze this movement data to determine alignment of user body positions by comparing current postures against reference positions. Based on this analysis, the system generates real-time feedback identifying specific areas requiring improvement such as foot placement, hip alignment, spinal extension, and arm engagement. Augmented reality (AR) glasses present spatially-aligned guidance to the user, including visual elements like a virtual instructor demonstrating proper form, alignment guides overlaid on the user's body, and highlighted areas requiring attention. The system may also provide audio cues for correcting form, track progress over time, and adjust difficulty levels based on user performance.

### CROSS-REFERENCE TO RELATED APPLICATION

This patent application claims the benefit of U.S. Provisional Patent Application No. 63/805,421, filed May 14, 2025, entitled “REAL-TIME BODY ALIGNMENT GUIDANCE SYSTEM”, which is incorporated by reference herein in its entirety.

### TECHNICAL FIELD

The present disclosures relate to augmented reality (AR) and artificial intelligence (AI) technologies and, in some examples, to algorithms and systems for providing real-time spatial feedback on physical activities using AR glasses, mobile body tracking, and large language models (LLMs).

### BACKGROUND

The field of augmented reality (AR) and wearable technology has seen rapid advancements in recent years, particularly in applications that enhance user experiences through interactive and immersive environments. AR systems are increasingly being integrated with artificial intelligence (AI) and machine learning (ML) to provide dynamic and responsive interactions. These technologies are being applied across various domains, including fitness, education, entertainment, and professional training, to create personalized and adaptive experiences. The use of wearable devices, such as AR glasses, has enabled the overlay of digital information onto the physical world, allowing users to engage with virtual elements in real-time.

### BRIEF DESCRIPTION OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some non-limiting examples are illustrated in the figures of the accompanying drawings in which:

FIGS. 1A-1G are interface diagrams illustrating a user interaction flow and device placement for a physical activity guidance system, according to some examples.

FIG. 2 is a screen view illustrating a pose-holding instruction interface displayed through augmented reality glasses during a physical activity session, according to some examples.

FIG. 3 is a diagram illustrating real-time feedback and alignment guidance for a physical pose with detailed improvement suggestions, according to some examples.

FIG. 4 is a user interface diagram illustrating initial setup options and menu selections for a physical activity guidance application, according to some examples.

FIG. 5 is a set of interface views illustrating pose tracking and feedback during exercise execution, according to some examples.

FIG. 6 is a feedback interface diagram illustrating focus areas and corrective guidance for user pose alignment, according to some examples.

FIG. 7 is a session summary interface illustrating an end-of-session overview with performance feedback, according to some examples.

FIG. 8 is a block diagram illustrating a networked environment in which the present disclosure may be deployed, according to some examples.

FIG. 9 is a system architecture diagram showing a layered view of an AI-integrated system, according to some examples.

FIG. 10 is a system diagram illustrating a system in which a head-wearable apparatus may be implemented, according to some examples.

FIG. 11 is a block diagram illustrating a software architecture within which examples may be implemented, according to some examples.

FIG. 12 is a schematic diagram illustrating a machine in the form of a computer system within which instructions may be executed, according to some examples.

### DETAILED DESCRIPTION

Physical activity guidance systems face technical challenges in providing effective instruction and feedback. Some video-based solutions rely on pre-recorded content that users must manually follow along with while checking their form. Mixed reality applications can demonstrate proper form through 3D models but lack the ability to monitor and assess user performance.

The technical limitations include capturing accurate body positioning data, analyzing complex physical movements in real-time, and delivering timely guidance that users can easily understand and follow. Existing approaches often require users to repeatedly reference screens or displays, breaking their focus and flow during practice.

Computer vision and motion tracking present additional technical hurdles in accurately detecting and measuring human body positions and movements across different environments and lighting conditions. Processing and analyzing this data requires significant computational resources while maintaining low latency for real-time feedback.

The field encompasses multiple technical domains including computer vision, artificial intelligence, augmented reality displays, wireless communications, and distributed computing systems working together to enable interactive physical activity guidance.

Physical activity guidance systems can benefit from technological advances in augmented reality (AR) and artificial intelligence. The described examples address limitations in existing solutions by combining mobile tracking, AR displays, and AI-powered analysis.

A mobile device may be positioned to capture the user's movements during physical activities, such as yoga. The mobile device can utilize its camera and on-device body tracking capabilities to analyze the user's form and positioning. This movement data may be transmitted to both AR glasses and AI processing systems for real-time analysis.

The AR glasses may present guidance through multiple modalities. Visual elements can include a virtual instructor demonstrating proper form, alignment guides overlaid on the user's body, and highlighted areas requiring attention. Audio cues may provide verbal instructions and corrections. When a user turns away from the virtual instructor, the display can automatically adjust to maintain visibility of key guidance elements.

An AI system incorporating large language models (LLMs) and computer vision can analyze the user's movements. The system may compare current positioning against reference poses, identify specific areas for improvement, and generate detailed feedback. This can include guidance on foot placement, hip alignment, spinal extension, and muscle engagement.

The system architecture may include multiple coordinated components. A client layer can handle user interactions through mobile apps and web interfaces. An API gateway layer may manage authentication, rate limiting, and request routing. The application layer can coordinate data flow while an AI service layer orchestrates multiple AI models for analysis.

The wearable apparatus may incorporate various technical elements. These can include an optical assembly for AR display, image processing capabilities, and both high-speed and low-power circuitry. The apparatus may communicate with mobile devices and server systems through wireless networking.

During operation, users may first position their mobile device to capture the user's full body. The system can display a camera frustum to guide proper placement. As users perform physical activities, they may receive real-time feedback through the AR glasses. The system can track pose hold duration and stability while providing timing guidance.

The software architecture may support multiple applications and frameworks. This can include dedicated components for location services, messaging, and third-party integration. The system may utilize various libraries and APIs while managing hardware resources through the operating system layer.

For data processing and storage, the system may employ a distributed architecture. Server systems can handle API requests, web services, and database operations. Third-party servers may provide additional capabilities through network integration.

The hardware architecture may include processors, memory systems, and I/O components. Communication modules can enable interaction with external devices and networks. The system may utilize both main memory and static memory for instruction processing.

Session data may be recorded and analyzed to track progress over time. Users can receive session summaries highlighting strengths and areas for improvement. The system may adjust difficulty levels based on user performance while maintaining engagement through features like progress tracking.

The integration of mobile tracking, AR visualization, and AI analysis can provide hands-free, immersive guidance that adapts to individual users and may enable more effective learning and practice of physical activities.

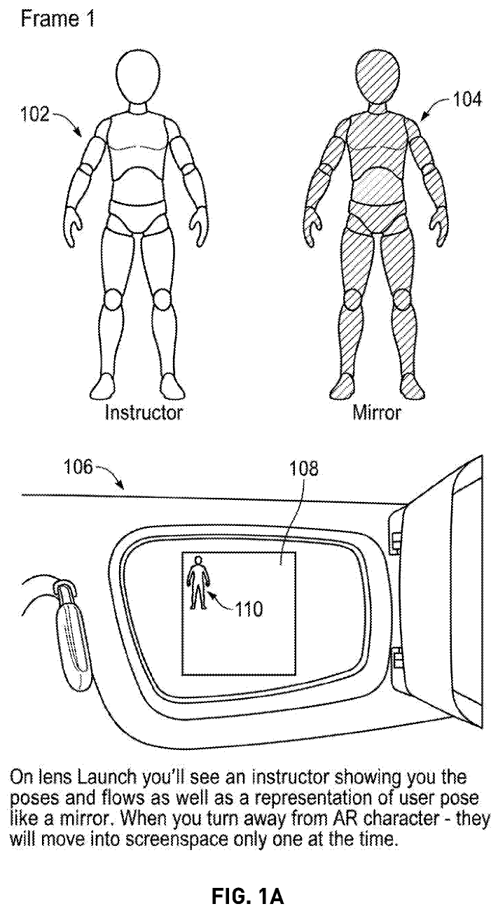

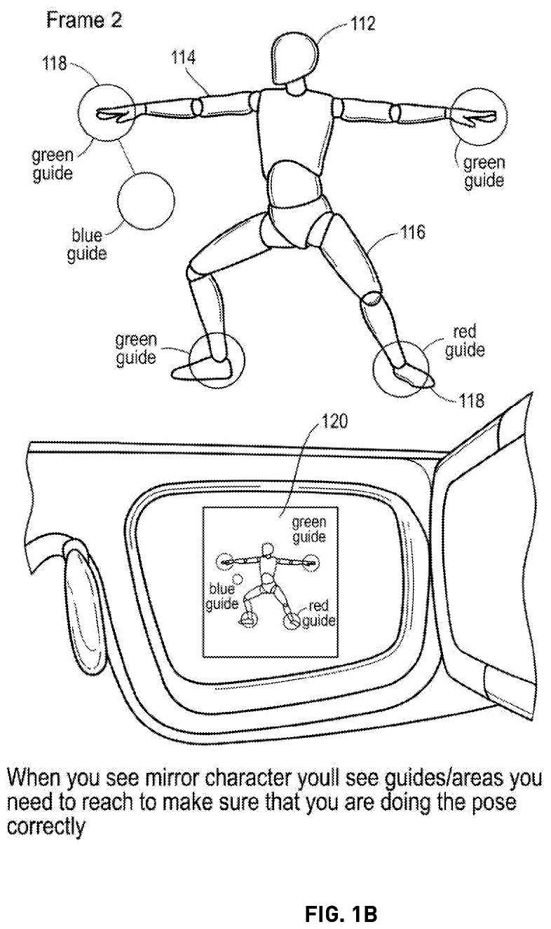

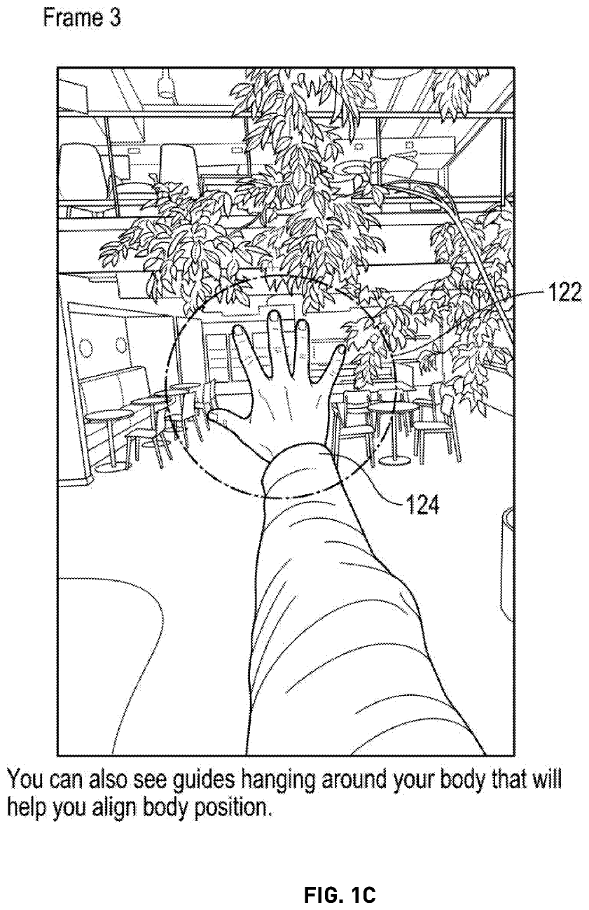

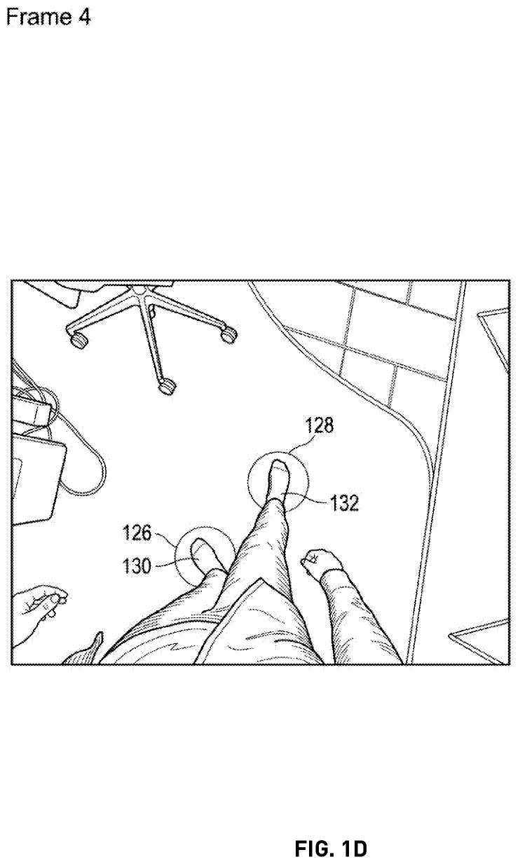

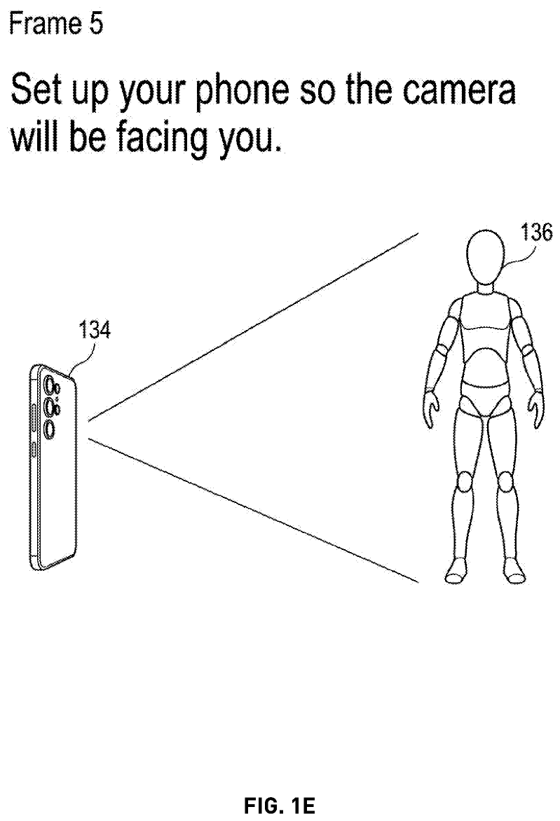

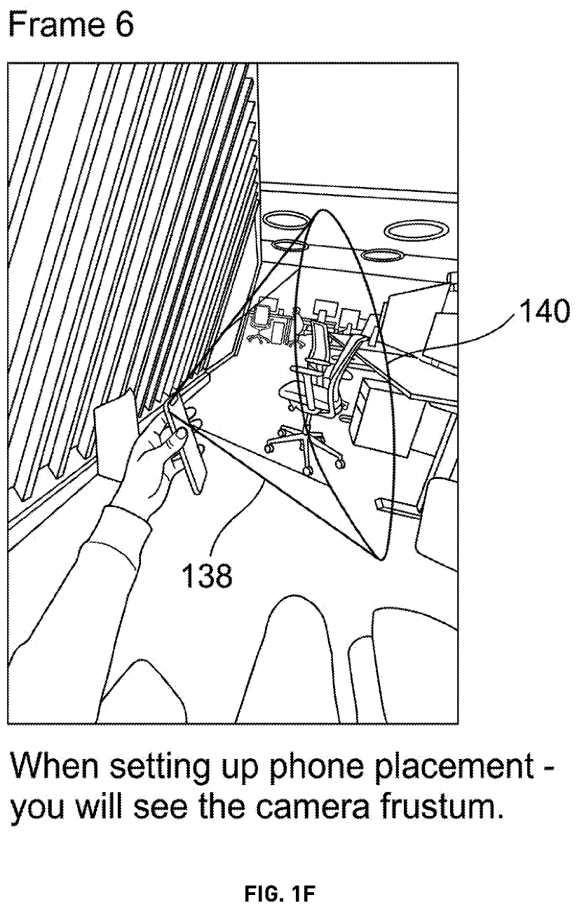

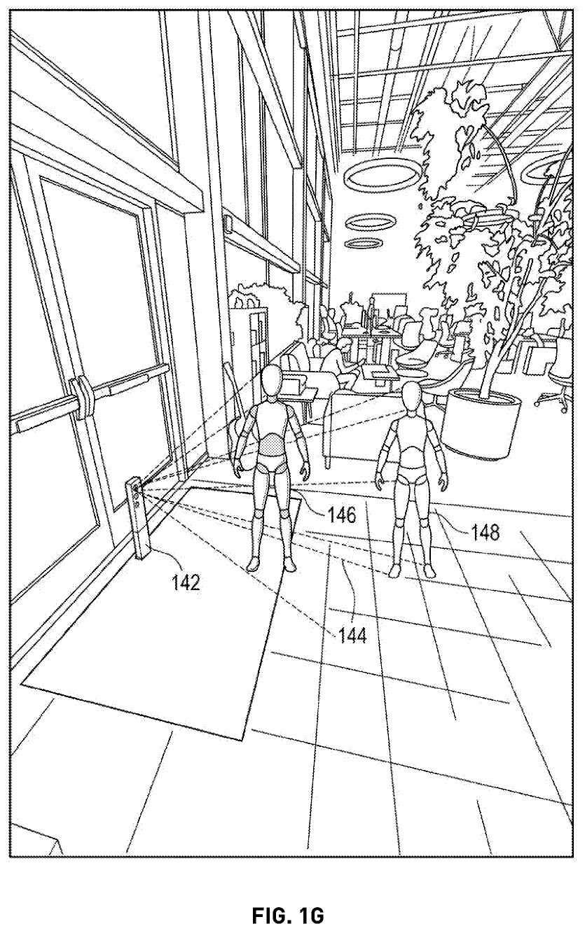

FIGS. 1A-1G illustrate examples of a user interaction flow and device placement for a physical activity guidance system, comprising several stages including a user flow, a setup stage, a guidance stage, a feedback stage, a placement flow, and a camera frustum visualization. These stages collectively demonstrate the process of configuring the system, receiving real-time guidance, and obtaining feedback during physical activity sessions.

FIG. 1A illustrates an example of an initial launch view presented through augmented reality (AR) glasses. As shown in FIG. 1A, an instructor character 102 and a mirror character 104 representing the user's pose may be presented to the user. A reflective or display surface 106 of a device may include a display area 108 within which an AR character 110 is rendered. The AR character 110 may mirror the pose of the user, providing a visual reference of the user's current body position. When the user turns away from the AR character, the displayed elements may move into screen space and may be presented one at a time to maintain visibility of guidance content.

FIG. 1B illustrates an example of alignment guides associated with the mirror character. As shown in FIG. 1B, a character 112 representing the user is depicted with arms 114 and legs 116. Guides 118 (which may be color-coded, for example, as green guides, blue guides, and red guides) may be associated with body parts of the character 112 to indicate target positions or areas the user is to reach in order to perform the pose correctly. A display 120 may present the character together with the guides 118, enabling the user to compare the user's current pose to the indicated target positions. A green guide may indicate that the user's pose of the relevant body part is correct, a red guide may indicate that the user's pose of the relevant body part is incorrect, and a blue guide may indicate the next pose of the relevant body part.

FIG. 1C illustrates an example of body-aligned guides presented within the user's field of view. As shown in FIG. 1C, one or more guides 122 may be rendered around the user's body, and a boundary or target area (for example, a circular boundary) may be displayed to assist the user in aligning a body part 124, such as a hand, with a target position. These guides assist the user in aligning body position during the activity.

FIG. 1D illustrates an example of floor-based positioning indicators. As shown in FIG. 1D, markers 126 and 128 may be presented on or relative to the floor in proximity to the user's feet 130 and 132. The markers 126, 128 may indicate target foot positions for a particular pose or may be used to determine user orientation and position within a physical space.

FIG. 1E illustrates an example of an instruction for positioning a mobile device for capture. As shown in FIG. 1E, a mobile device 134, such as a smartphone, may be positioned such that its camera faces a representation of the user 136. The user is instructed to set up the mobile device so that the camera is facing the user, allowing the camera to capture images of the user during the physical activity session.

FIG. 1F illustrates an example of a camera frustum visualization presented during phone placement. As shown in FIG. 1F, a user may hold a mobile device 138, and a camera frustum 140 may be presented as a visual overlay representing the field of view of the camera of the mobile device. The camera frustum 140 may guide the user in positioning the mobile device such that the user's body is contained within the field of view of the camera, supporting accurate tracking and analysis.

FIG. 1G illustrates an example of a placement environment with avatars positioned relative to a placement region. As shown in FIG. 1G, a placement region 142, such as a mat or sensor area, may be located on the floor. One or more avatars 146, 148 may be positioned at or near the placement region 142, and a boundary 144, which may be represented by a dashed line, may indicate an interaction zone or path of movement associated with the avatars and the placement region 142.

The user flow stage represents the initial interaction with the system, including the launch process and the visualization of the user's body, for example as a skeletal representation. This visualization reflects capture and modeling of the user's body positions using the mobile device's camera and body tracking capabilities. The skeletal representation may serve as a basis for subsequent analysis and feedback.

The setup stage may involve a calibration phase, where the user adjusts the user's position and posture to align with the system's requirements. During this stage, the system may detect and analyze the user's body alignment, supporting accurate tracking and preparation for the activity session. Visual cues may be provided to assist the user in achieving the setup.

The guidance stage provides real-time support to the user during the physical activity. This may include visual overlays, such as alignment guides and virtual instructor demonstrations, presented through augmented reality glasses. The system may dynamically adjust the guidance based on the user's movements, providing continuous support and correction throughout the session.

The feedback stage evaluates the user's performance and delivers detailed insights. This may include visual indicators highlighting areas for improvement, textual instructions for adjustments, and audio cues for corrections. The feedback may help the user refine the user's form during the session.

The placement flow stage demonstrates the process of positioning the mobile device to capture the user's movements. Instructions may be provided to set up the device with the camera facing the user, supporting visibility and tracking accuracy.

The camera frustum visualization stage provides a graphical representation of the camera's field of view during the setup phase. This visualization may guide the user in positioning the mobile device such that the user's body is visible within the field of view for tracking and analysis.

The arrangements depicted in FIGS. 1A-1G are provided as non-limiting examples. Other arrangements, sequences, and combinations of the depicted stages, characters, guides, markers, and visual elements may be used. For example, one or more of the user flow stage, the setup stage, the guidance stage, the feedback stage, the placement flow stage, and the camera frustum visualization stage may be omitted, combined, reordered, or supplemented with additional stages without departing from the scope of this disclosure.

Although FIGS. 1A and 1B depict humanoid mannequin-style representations for the instructor character and the mirror character, the instructor and mirror characters may take any suitable form, including, by way of non-limiting example, stylized avatars, photorealistic avatars, skeletal representations, two-dimensional figures, three-dimensional models, animated characters, or abstract graphical representations. Similarly, although certain guides 118 are depicted in association with particular body parts and described with reference to colors such as green, blue, and red, the guides may be presented using any suitable visual attributes, including but not limited to other colors, shapes, sizes, line styles, animations, opacity levels, or textual labels, and may be associated with any body parts or regions of interest.

The reference numerals shown in FIGS. 1A-1G are used to identify particular components and elements for purposes of description. The depicted components are not limited to the specific shapes, proportions, or spatial arrangements shown. Additional components may be present, and certain components depicted may be omitted in some examples. The pose shown in FIG. 1B is one example of a pose; the system may guide users through any number of poses, including yoga poses, fitness exercises, dance movements, rehabilitation exercises, or other physical activities.

The placement region 142 depicted in FIG. 1G may include any suitable physical or virtual region, such as a yoga mat, an exercise mat, a designated floor area, a sensor pad, or a virtually defined area within a physical environment. The boundary 144 may be presented using any suitable visual representation, including dashed lines, solid lines, shaded regions, or three-dimensional volumes, and may indicate an interaction zone, a tracking boundary, a calibration area, a path of movement, or a safety boundary. The avatars 146, 148 may represent users, instructors, virtual coaches, other participants, or any combination thereof.

The mobile device 134 depicted in FIG. 1E and the device held in FIG. 1F may include any suitable computing device having a camera, including but not limited to a smartphone, a tablet, a laptop computer, a dedicated camera device, a smart home device with a camera, or any other device capable of capturing images of the user. The camera frustum 140 may be presented in any suitable form, including a wireframe representation, a shaded volume, a translucent overlay, or a two-dimensional projection, and may be presented through the AR glasses, on a display of the mobile device, or through any combination of devices.

Although certain views in FIGS. 1A-1G are depicted from particular perspectives, including a third-person view in FIG. 1A and a first-person view in FIG. 1C, the views may be presented from any suitable perspective. The visual elements depicted in FIGS. 1A-1G may be presented through AR glasses, virtual reality headsets, mobile device displays, computer monitors, projection systems, or any combination thereof.

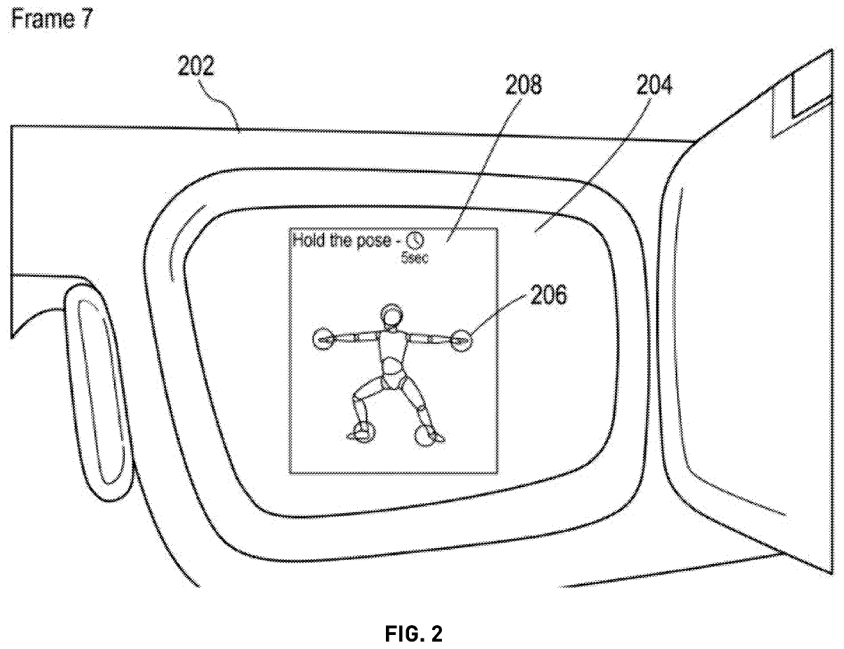

FIG. 2 illustrates an example of a user interface displayed through augmented reality (AR) glasses during a physical activity session. As shown in FIG. 2, the user interface may include a display area 202 and a region 204 within the display area 202. A graphical representation 206 of a user or a target pose may be presented within the region 204. A visual indicator 208 may instruct the user to hold a specific pose for a designated duration, for example, by presenting text such as “Hold the pose” together with a duration such as “5 sec.” The visual indicator 208 may provide real-time guidance and feedback to the user, supporting hands-free interaction with the physical activity guidance system.

The visual indicator 208 may be accompanied by graphical representations of the user's body, including highlighted joints and segments. These graphical elements may be color-coded to indicate alignment and engagement, assisting the user in understanding the pose and maintaining form during the activity.

The graphical representation 206 of the user's body may be generated based on movement data captured by a mobile device. The representation 206 may include skeletal models and positional markers that reflect the user's current posture and alignment. The system may dynamically update the visual elements to provide responsive feedback, allowing the user to make real-time adjustments to the user's form.

The AR glasses may present the visual indicator 208 and graphical elements by spatially aligning the content with the user's perspective. This alignment may support visibility of the guidance as the user moves or changes orientation. Presenting guidance through AR glasses may allow the user to focus on the activity without referring to external screens or devices.

The arrangement depicted in FIG. 2 is provided as a non-limiting example. Other arrangements, layouts, and combinations of the visual indicator 208, the region 204, and the graphical representation 206 may be used. The display area 202, the region 204, the graphical representation 206, and the visual indicator 208 may be presented in any suitable position, size, shape, or visual style, and one or more of these elements may be omitted, combined, or supplemented with additional elements.

Although FIG. 2 depicts a designated duration of five seconds, the duration may be any suitable length of time, including durations shorter or longer than five seconds, and may be fixed, user-configurable, or dynamically determined based on the pose, the user's skill level, prior performance, or other factors. The visual indicator 208 may present the duration in any suitable form, including a numerical countdown, a progress bar, a circular timer, an animated graphic, or any combination thereof, and may be supplemented or replaced by audio cues, haptic feedback, or other modalities.

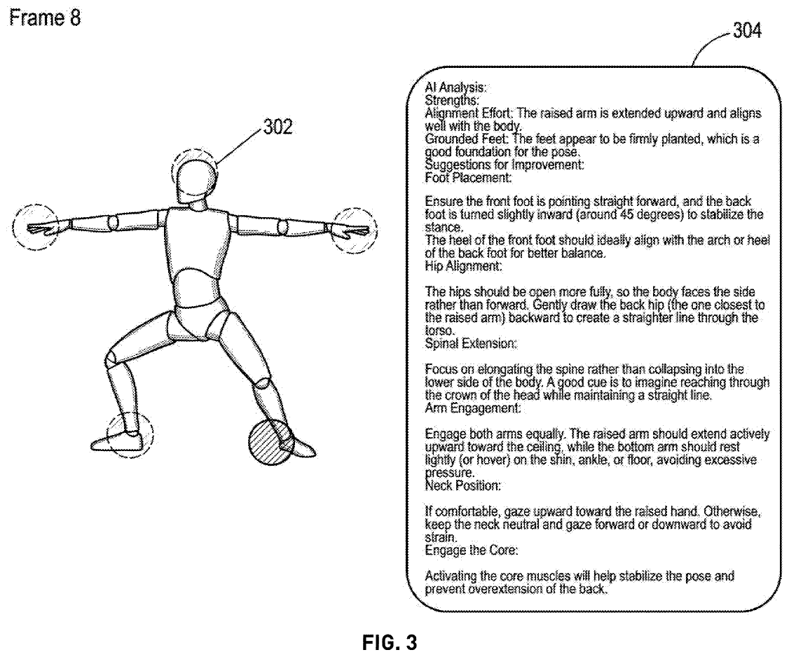

FIG. 3 illustrates an example of a physical activity guidance system that delivers real-time feedback and alignment cues during a physical activity session. As shown in FIG. 3, a representation 302 of the user in a pose may be presented, with one or more body parts highlighted to indicate points of attention. A feedback element 304, such as a text box, may present an analysis of the pose, which may include identified strengths, suggestions for improvement, and specific instructions relating to one or more of foot placement, hip alignment, spinal extension, arm engagement, neck position, and core engagement. The system may employ augmented reality (AR) glasses to project visual indicators onto the user's field of view to assist the user in maintaining form and posture during the activity.

One visual indicator may represent the user's raised arm, which may be displayed as an extended arm aligned with the user's torso. The system may analyze the position of the arm to determine whether the arm is engaged and aligned with the user's body. Visual cues may be provided through the AR glasses to indicate misalignment, supporting steady positioning of the arm and contributing to posture stability.

Another visual indicator may highlight the user's front foot, which may be positioned forward to serve as an anchor point for the user's stance. The system may evaluate the placement and orientation of the front foot to determine whether the front foot is planted and aligned with the user's body. Feedback may be provided to guide the user in adjusting the position of the foot to support balance and stability.

The system may also monitor the user's back foot, which may be positioned behind the user and angled to support the stance. The system may evaluate the angle and placement of the back foot and determine whether the back foot complements the positioning of the front foot. Visual indicators may guide the user in refining the placement of the back foot to support posture and weight distribution.

Spinal alignment may be addressed by the system. The system may analyze the user's spinal position to determine whether the spine is extended and aligned with other portions of the body. Visual overlays may highlight areas for adjustment, assisting the user in achieving a straight spine.

The system may indicate engagement of the user's central muscle groups to stabilize the pose and reduce overextension of the back. Feedback may be provided regarding muscle activation, with visual and textual cues guiding the user in maintaining engagement. This may contribute to form and balance during the activity.

By integrating these visual indicators and feedback mechanisms, the system may support the user in sustaining form and posture during the physical activity session. Use of the AR glasses may allow the guidance to be spatially aligned with the user's perspective, supporting hands-free interaction.

The arrangement depicted in FIG. 3 is provided as a non-limiting example. Other arrangements, layouts, and combinations of the representation 302 and the feedback element 304 may be used. The representation 302 may take any suitable form, including a stylized humanoid figure, a skeletal model, a wireframe representation, a photorealistic avatar, a two-dimensional figure, a three-dimensional model, or any combination thereof, and may be presented from any suitable perspective. The highlighted body parts depicted in FIG. 3 are provided as examples; any body parts or regions of interest may be highlighted using any suitable visual attributes, including colors, shapes, sizes, line styles, animations, opacity levels, or textual labels.

The feedback element 304 may be presented in any suitable form, including a text box, a speech bubble, a callout, an overlay, a panel, an audio output, a haptic output, or any combination thereof. The content of the feedback element 304 is provided as an example and is not limited to the specific categories of strengths, suggestions for improvement, foot placement, hip alignment, spinal extension, arm engagement, neck position, or core engagement. The feedback element 304 may include any suitable feedback content, including evaluations of any body parts, joints, or movement attributes, and may be generated by one or more artificial intelligence models, rule-based systems, or any combination thereof.

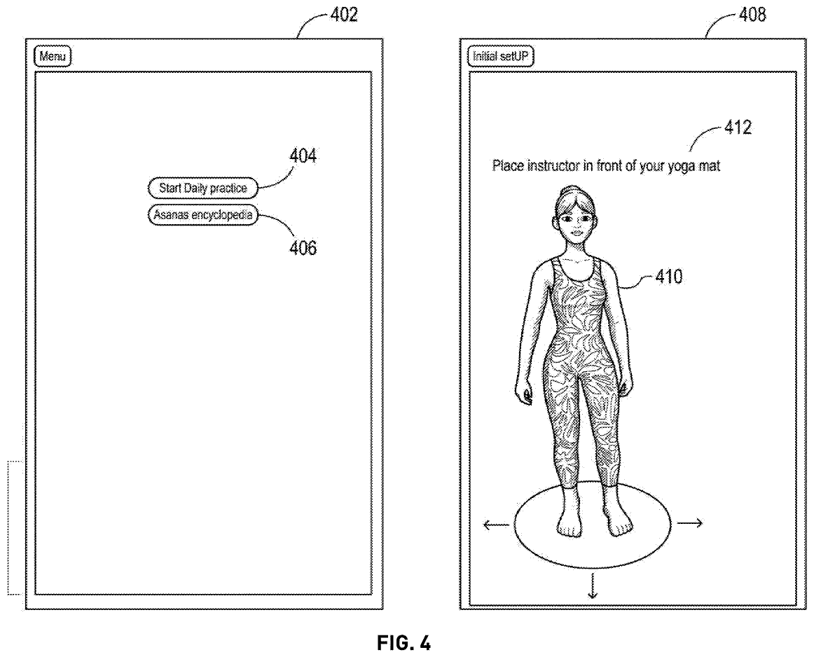

FIG. 4 illustrates an example of a user interface for a physical activity guidance application. As shown in FIG. 4, a menu interface 402 may present a menu including a selectable option 404 labeled “Start Daily practice” and a selectable option 406 labeled “Asanas encyclopedia.” The initial setup interface 408 may provide the user with a visual representation of the placement of the representation 410 of the instructor relative to a yoga mat. This view may support the positioning of the virtual instructor to provide visibility and alignment during the session. The system may use visual cues, such as arrows and a highlighted area surrounding the feet of the representation 410, to guide the user in achieving placement.

A textual instruction 412 may guide the user to “Place instructor in front of your yoga mat.” The user interface depicted in FIG. 4 demonstrates initial setup and menu options available to the user for engaging with the system.

The selectable option 404 labeled “Start Daily practice” may allow the user to initiate a daily practice session. This option may provide a pathway to begin guided physical activities. The system may customize the session based on user preferences and previous progress, including options such as initial setup, menu navigation, instructor placement, and access to additional resources.

The selectable option 406 labeled “Asanas encyclopedia” may offer the user an option to access additional resources and information related to physical activities. This option may provide content, including explanations of poses, techniques, and benefits.

The arrangement depicted in FIG. 4 is provided as a non-limiting example. The interface 402, the selectable options 404 and 406, the interface 408, the representation 410 of the instructor, and the textual instruction 412 may be presented in any suitable position, size, shape, layout, or visual style, and one or more of these elements may be omitted, combined, reordered, or supplemented with additional elements. The selectable options 404 and 406 are not limited to the labels “Start Daily practice” and “Asanas encyclopedia”; any suitable selectable options may be presented, including options corresponding to different practice styles, difficulty levels, focus areas, duration preferences, instructional content, or any combination thereof. The representation 410 of the instructor may take any suitable form, including a stylized humanoid figure, a skeletal model, a photorealistic avatar, a two-dimensional figure, a three-dimensional model, or any combination thereof, and may correspond to a yoga instructor, a fitness trainer, a virtual coach, or any other guide. The textual instruction 412 may be supplemented or replaced by audio cues, haptic feedback, animations, or other modalities, and is not limited to placement relative to a yoga mat; the instruction may relate to placement relative to any suitable physical or virtual reference, including an exercise mat, a designated floor area, a sensor pad, or a virtually defined region.

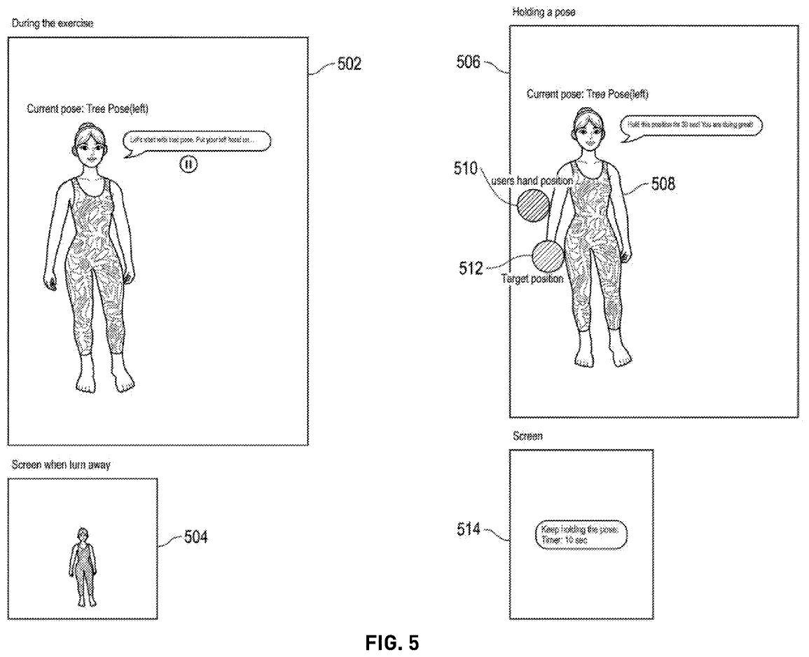

FIG. 5 illustrates examples of user interface views for a physical activity guidance system, according to some examples. As shown in FIG. 5, the system may present an interface 502 during an exercise and a screen 504 when the user turns away from a primary view. The interface 502 may include a representation 508 of the user in a pose, identified for example as “Tree Pose (left).” An interface 506 may indicate that a pose is being held. The interface 506 may include a circle 510 representing the user's current hand position and a circle 512 representing a target hand position for the pose. A screen 514 may present a “Keep holding the pose” message and a timer indicating a remaining duration. The screen 504 may present a reduced view, such as a miniature representation of the user, when the user turns away from the primary display.

In some examples, the interface 502 may present directional indicators, such as red arrows, overlaid on the representation 508 to indicate movement paths or areas for adjustment. The directional indicators may serve as visual cues to guide the user in refining the user's posture or transitioning to an alignment. The screen 504 may demonstrate adaptive display capabilities of the system, supporting visibility of guidance elements when the user turns away from the primary display or changes orientation during the activity.

In some examples, the interface 506 may present circular indicators, including the circle 510 and the circle 512, positioned at body locations such as joints or segments to indicate positioning and alignment points. The system may use these circular indicators to assist the user in achieving form during the pose. For example, the circle 510 and the circle 512 may correspond to alignment of the spine, positioning of the arms, or placement of the feet. The interface 506 may dynamically update the circular indicators based on the user's movements.

The interfaces 502, 506 and the screens 504, 514 may demonstrate integration of the system with a mobile device acting as an external tracker. The mobile device may capture the user's body position using a camera and on-device body tracking capabilities. The captured data may be analyzed to generate real-time feedback, which may be presented in the user interface views. The visual guidance elements, including the circle 510 and the circle 512, may be spatially aligned with the user's body to provide positioning cues. This alignment may allow the user to make adjustments while focusing on the activity.

Adaptive display features and spatially aligned feedback presented through the interfaces 502, 506 and the screens 504, 514 may support hands-free guidance. By using augmented reality overlays and mobile tracking data, the system may support the user in maintaining form, refining posture, and progressing through poses. Visual cues, adaptive display features, and real-time analysis may be applied to physical activities including yoga, fitness exercises, dance, rehabilitation, and other activities.

The arrangement depicted in FIG. 5 is provided as a non-limiting example. The interfaces 502, 506, the screens 504, 514, the representation 508, the circle 510, the circle 512, and other elements may be presented in any suitable position, size, shape, layout, or visual style, and one or more of these elements may be omitted, combined, reordered, or supplemented with additional elements. The pose identified as “Tree Pose (left)” is provided as one example of a pose; the system may guide the user through any number of poses, including yoga poses, fitness exercises, dance movements, rehabilitation exercises, or other physical activities. The circle 510 and the circle 512 are not limited to the hand and may correspond to any body part, joint, or region of interest. The directional indicators, circular indicators, and timer may be presented using any suitable visual attributes, including any colors, shapes, sizes, line styles, animations, opacity levels, or textual labels, and may be supplemented or replaced by audio cues, haptic feedback, or other modalities. The screen 504 may present any suitable reduced or alternative view, including a miniature representation, a simplified avatar, an icon, a status indicator, or any combination thereof, when the user turns away from a primary display or changes orientation.

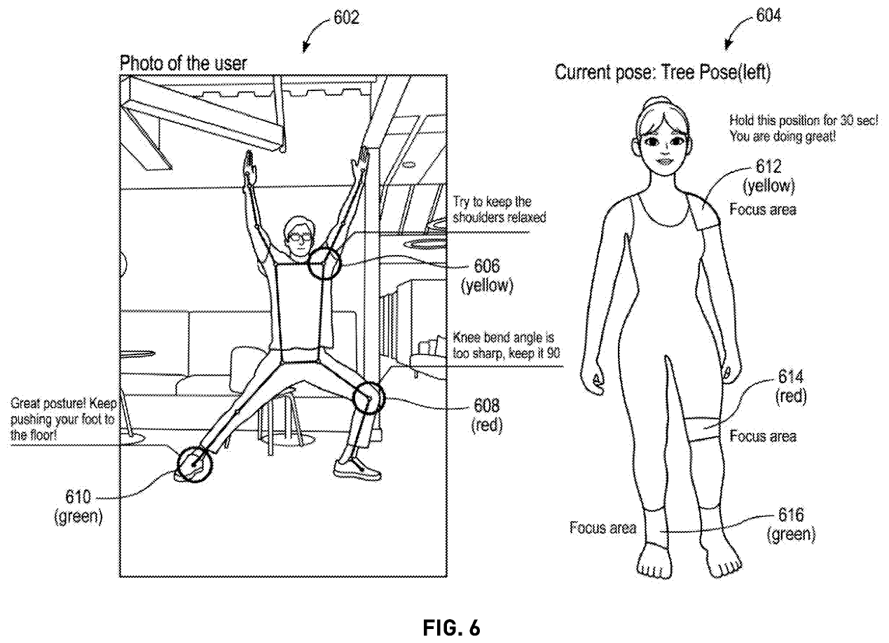

FIG. 6 illustrates examples of visual elements of a user interface for a physical activity guidance system, demonstrating integration of real-time feedback and augmented reality overlays to assist the user in maintaining form and alignment during physical activities. As shown in FIG. 6, the user interface may include a photo of the user 602 and a representation 604 of a target pose, such as “Tree Pose (left).”

The photo of the user 602 may be captured by a camera of a mobile device. The photo of the user 602 may include circular markers overlaid on body positions, such as joints and limbs, to indicate areas for attention or adjustment. These circular markers may correspond to focus areas that the system evaluates for alignment, stability, and engagement. For example, a shoulder indicator 606 may be associated with a shoulder of the user, a knee bend angle indicator 608 may indicate a measured knee bend angle, and a foot indicator 610 may be associated with a foot of the user. The system may dynamically update the markers based on the user's movements. In some examples, the shoulder indicator 606 may be presented in a first color (for example, yellow), the knee bend angle indicator 608 may be presented in a second color (for example, red) to indicate that the angle is outside a target range, and the foot indicator 610 may be presented in a third color (for example, green) to indicate that the foot position is within a target range. Textual feedback may be presented in association with the markers, including, for example, an instruction to maintain shoulder relaxation, an instruction that a knee bend angle is outside a target range, or an indication that a foot position is within a target range.

The representation 604 may include a graphical model of a virtual figure that visually corresponds to the focus areas identified in association with the photo of the user 602. The representation 604 may serve as a reference, presenting focus areas as circular indicators to demonstrate form and alignment. For example, a shoulder indicator 612 may be associated with a shoulder of the representation 604, a knee indicator 614 may be associated with a knee of the representation 604, and an ankle indicator 616 may be associated with an ankle of the representation 604. In some examples, the shoulder indicator 612 may be presented in a first color (for example, yellow), the knee indicator 614 may be presented in a second color (for example, red), and the ankle indicator 616 may be presented in a third color (for example, green). The representation 604 may include visual cues, such as highlighted segments and alignment guides, to assist the user in understanding how the user's posture compares to a target pose. The focus areas on the representation 604 may be spatially aligned with focus areas associated with the photo of the user 602.

By presenting both the photo of the user 602 and the representation 604, the system may allow the user to compare the user's current posture to a reference pose in real time. The integration of circular markers and focus areas across the photo of the user 602 and the representation 604 may support clear feedback, allowing the user to make adjustments to the user's form. This dual-view interface may support refinement of physical activity performance through visual and spatially aligned guidance.

The arrangement depicted in FIG. 6 is provided as a non-limiting example. The photo of the user 602, the representation 604, the shoulder indicator 606, the knee bend angle indicator 608, the foot indicator 610, the shoulder indicator 612, the knee indicator 614, the ankle indicator 616, and other elements may be presented in any suitable position, size, shape, layout, or visual style, and one or more of these elements may be omitted, combined, reordered, or supplemented with additional elements. The pose identified as “Tree Pose (left)” is provided as one example of a pose; the system may guide the user through any number of poses, including yoga poses, fitness exercises, dance movements, rehabilitation exercises, or other physical activities. The indicators 606, 608, 610, 612, 614, and 616 are not limited to the specific body parts identified and may correspond to any body part, joint, or region of interest. Although certain indicators are described with reference to colors such as yellow, red, and green, the indicators may be presented using any suitable visual attributes, including any colors, shapes, sizes, line styles, animations, opacity levels, or textual labels, and the color associations described are provided as examples and are not limiting. The textual feedback associated with the indicators is provided as an example and may be replaced or supplemented by any suitable feedback content, audio cues, haptic feedback, or other modalities. The representation 604 may take any suitable form, including a stylized humanoid figure, a skeletal model, a wireframe representation, a photorealistic avatar, a two-dimensional figure, a three-dimensional model, or any combination thereof. The photo of the user 602 may be replaced or supplemented by any suitable representation of the user, including a live video feed, a real-time skeletal overlay, an avatar tracking the user's movements, or any combination thereof.

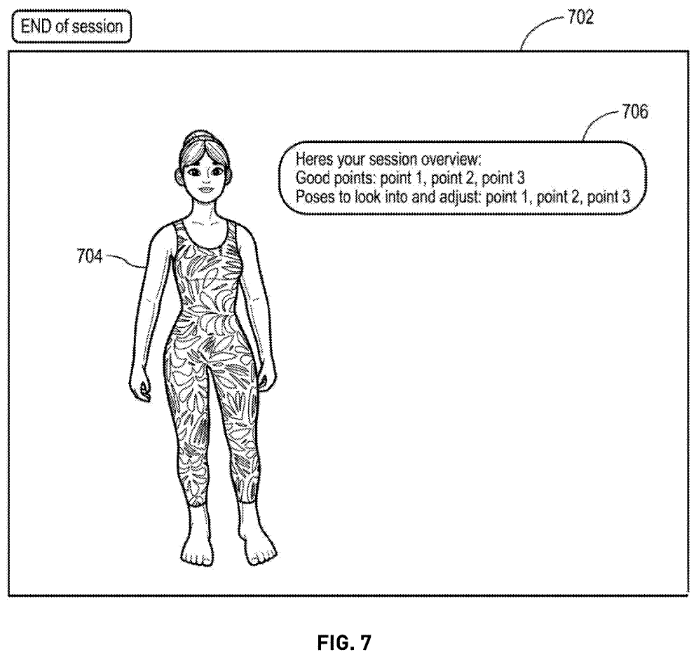

FIG. 7 illustrates an example of a user interface displayed at the conclusion of a physical activity session. As shown in FIG. 7, the user interface 702 may include a representation 704 of the user in a pose and a text element 706, such as a text bubble or annotation box, presenting session feedback. The user interface may provide visual and textual elements to summarize the user's performance and offer guidance for future sessions.

A visual indicator may be presented within or in association with the interface 702 to signify the end of the session. This element may mark a transition from active guidance to post-session feedback, indicating that the activity has concluded and that the user may review the user's results.

The text element 706 may include a text-based summary of the session, which may provide feedback on the user's performance. The summary may include identified strengths, evaluations of posture, areas for adjustment, and recommendations for improvement. The textual feedback may offer insights to support the user in refining technique and physical activity practice in subsequent sessions.

The user interface 702 may combine visual and textual elements, including the representation 704 and the text element 706, to provide a post-session experience supporting the user in tracking progress and identifying areas for refinement.

The arrangement depicted in FIG. 7 is provided as a non-limiting example. The interface 702, the representation 704, the text element 706, and other elements may be presented in any suitable position, size, shape, layout, or visual style, and one or more of these elements may be omitted, combined, reordered, or supplemented with additional elements. The representation 704 may take any suitable form, including a stylized humanoid figure, a skeletal model, a wireframe representation, a photorealistic avatar, a two-dimensional figure, a three-dimensional model, or any combination thereof. The text element 706 may be presented in any suitable form, including a text bubble, an annotation box, a callout, an overlay, a panel, a list, a chart, or any combination thereof, and may be supplemented or replaced by audio cues, haptic feedback, animations, or other modalities. The session feedback content is provided as an example and is not limited to identified strengths, evaluations of posture, areas for adjustment, or recommendations for improvement; the feedback may include any suitable content, including performance metrics, comparisons to prior sessions, progress trends, suggested poses, or any combination thereof, and may be generated by one or more artificial intelligence models, rule-based systems, or any combination thereof. The user interface depicted in FIG. 7 may be presented through AR glasses, virtual reality headsets, mobile device displays, computer monitors, projection systems, or any combination thereof.

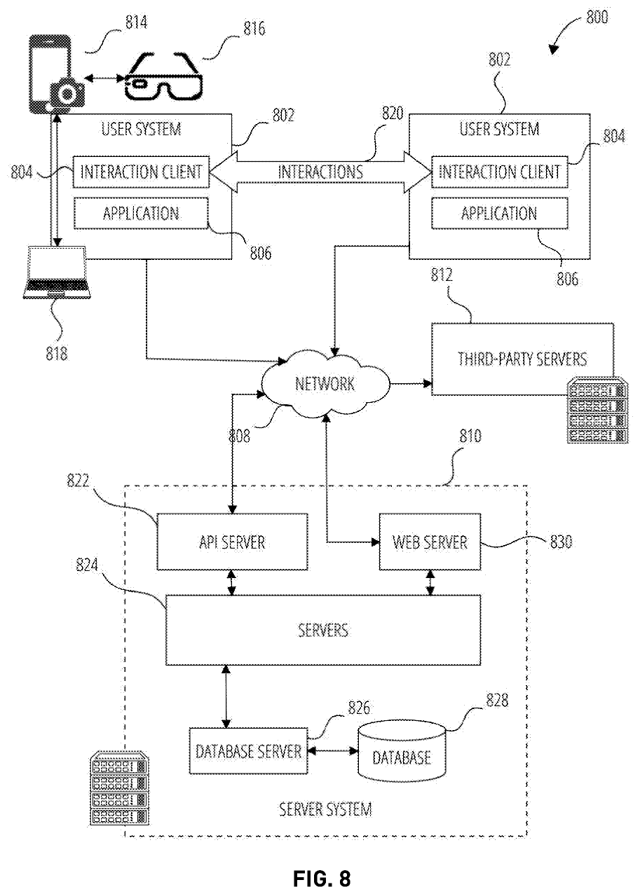

FIG. 8 is a block diagram showing an example digital interaction system 800 for facilitating physical activity guidance and movement analysis over a network. The digital interaction system 800 includes multiple user systems 802, each of which hosts multiple applications, including an interaction client 804 and other applications 806. Each interaction client 804 is communicatively coupled, via one or more networks including a Network 808 (e.g., the Internet), to other instances of the interaction client 804 (e.g., hosted on respective other user systems 802), a server system 810 and third-party servers 812). An interaction client 804 can also communicate with locally hosted applications 806 using Applications Program Interfaces (APIs).

Each user system 802 may include multiple user devices, such as a mobile device 814, wearable apparatus 816, and a computer client device 818 that are communicatively connected to exchange data and messages. The mobile device 814 serves as an external body tracker during physical activity sessions, while the wearable apparatus 816 may be, for example, AR glasses displaying real-time guidance, virtual reality (VR) headsets, fitness trackers, smartwatches, smart clothing, head-mounted displays (HMDs), hearing aids, smart helmets, wearable cameras, medical wearables, or earphones.

An interaction client 804 interacts with other interaction clients 804 and with the server system 810 via the Network 808. The data exchanged between the interaction clients 804 (e.g., interactions 820) and between the interaction clients 804 and the server system 810 includes functions (e.g., commands to invoke functions) and payload data (e.g., body tracking information, camera images, pose analysis data, alignment feedback, and other multimedia data).

The server system 810 provides server-side functionality via the Network 808 to the interaction clients 804. While certain functions of the digital interaction system 800 are described herein as being performed by either an interaction client 804 or by the server system 810, the location of certain functionality either within the interaction client 804 or the server system 810 may be a design choice. For example, it may be technically preferable to initially deploy particular technology and functionality within the server system 810 but to later migrate this technology and functionality to the interaction client 804 where a user system 802 has sufficient processing capacity. This flexibility is particularly valuable for balancing computational demands of physical activity analysis between cloud and edge devices.

The server system 810 supports various services and operations that are provided to the interaction clients 804. Such operations include transmitting data to, receiving data from, and processing data generated by the interaction clients 804. This data may include body pose information, joint position coordinates, movement trajectories, camera images, user profile data, session histories, reference poses, form analysis results, and performance metrics. Data exchanges within the digital interaction system 800 are invoked and controlled through functions available via user interfaces (UIs) of the interaction clients 804.

Turning now specifically to the server system 810, an Application Program Interface (API) server 822 is coupled to and provides programmatic interfaces to servers 824, making the functions of the servers 824 accessible to interaction clients 804, other applications 806 and third-party servers 812. The servers 824 are communicatively coupled to a database server 826, facilitating access to a database 828 that stores data associated with physical activity sessions processed by the servers 824. Similarly, a web server 830 is coupled to the servers 824 and provides web-based interfaces to the servers 824. To this end, the web server 830 processes incoming network requests over the Hypertext Transfer Protocol (HTTP) and several other related protocols.

The Application Program Interface (API) server 822 receives and transmits interaction data (e.g., commands and message payloads) between the servers 824 and the user systems 802 (and, for example, interaction clients 804 and other application 806) and the third-party server 812. Specifically, the Application Program Interface (API) server 822 provides a set of interfaces (e.g., routines and protocols) that can be called or queried by the interaction client 804 and other applications 806 to invoke functionality of the servers 824. The Application Program Interface (API) server 822 exposes various functions supported by the servers 824, including user account registration; login functionality; the transmission of body tracking data from a mobile device to the servers 824; the processing and analysis of physical movement data; the generation of form feedback and alignment guidance; the communication of media files (e.g., images or video) from an interaction client 804 to the servers 824; the storage and retrieval of session histories and performance metrics; the management of reference pose libraries; the tracking of user progress over time; and the coordination of shared physical activity sessions between multiple users.

The servers 824 host multiple systems and subsystems specialized for physical activity guidance, including:
1. AI Model Orchestration Services-Coordinate the execution of various AI models, including large language models (LLMs), computer vision models, and pose estimation models to analyze user movements and generate personalized feedback.
2. Movement Analysis Services-Process body tracking data to evaluate alignment, stability, and form during physical activities, comparing user positions against reference poses to identify deviations and areas for improvement.
3. Feedback Generation Services-Create detailed, contextually-relevant feedback based on movement analysis, including specific corrections for foot placement, hip alignment, spinal extension, and muscle engagement.
4. Session Management Services-Track session progress, manage pose sequences, and determine appropriate timing for transitions between poses based on user performance and stability.
5. User Profile Services-Maintain user preferences, physical characteristics, skill levels, and historical performance data to personalize guidance and track improvements over time.
6. Media Processing Services-Handle image and video data captured during physical activity sessions, processing visual inputs for analysis and feedback generation.
7. Spatial Registration Services-Ensure proper alignment of virtual guidance elements with the user's physical environment and body position, enabling accurate overlay of instructional content.
8. Progress Tracking Services-Analyze performance trends across multiple sessions, identify patterns in user form and alignment, and generate long-term improvement recommendations.
9. These services collectively enable the physical activity guidance system to deliver personalized, real-time feedback that helps users improve their form and technique during activities such as yoga and fitness exercises.

The interaction client 804 provides a user interface that allows users to access features and functions of an external resource, such as a linked application 806, an applet, or a microservice. This external resource may be provided by a third party or by the creator of the interaction client 804. For physical activity guidance, these external resources might include specialized fitness tracking applications, pose libraries, nutrition apps, or wellness platforms.

The external resource may be a full-scale application installed on the user's system 802, or a smaller, lightweight version of the application, such as an applet or a microservice, hosted either on the user's system or remotely, such as on third-party servers 812 or in the cloud. These smaller versions, which include a subset of the full application's features, may be implemented using a markup-language document and may also incorporate a scripting language and a style sheet.

When a user selects an option to launch or access the external resource, the interaction client 804 determines whether the resource is web-based or a locally installed application. Locally installed applications can be launched independently of the interaction client 804, while applets and microservices can be launched or accessed via the interaction client 804.

If the external resource is a locally installed application, the interaction client 804 instructs the user's system to launch the resource by executing locally stored code. If the resource is web-based, the interaction client 804 communicates with third-party servers to obtain a markup-language document corresponding to the selected resource, which it then processes to present the resource within its user interface.

The interaction client 804 can also notify users of activity in one or more external resources. For instance, it can provide notifications relating to scheduled physical activity sessions, remind users of daily practice goals, or alert users to new content from fitness instructors. Users can be invited to join group yoga sessions or participate in community challenges through these notifications.

The interaction client 804 can present a list of available external resources to a user, allowing them to launch or access a given resource. This list can be presented in a context-sensitive menu, with icons representing different applications, applets, or microservices varying based on how the menu is launched by the user. For physical activity guidance, this might include options for different practice styles, difficulty levels, focus areas (such as flexibility or strength), or duration preferences.

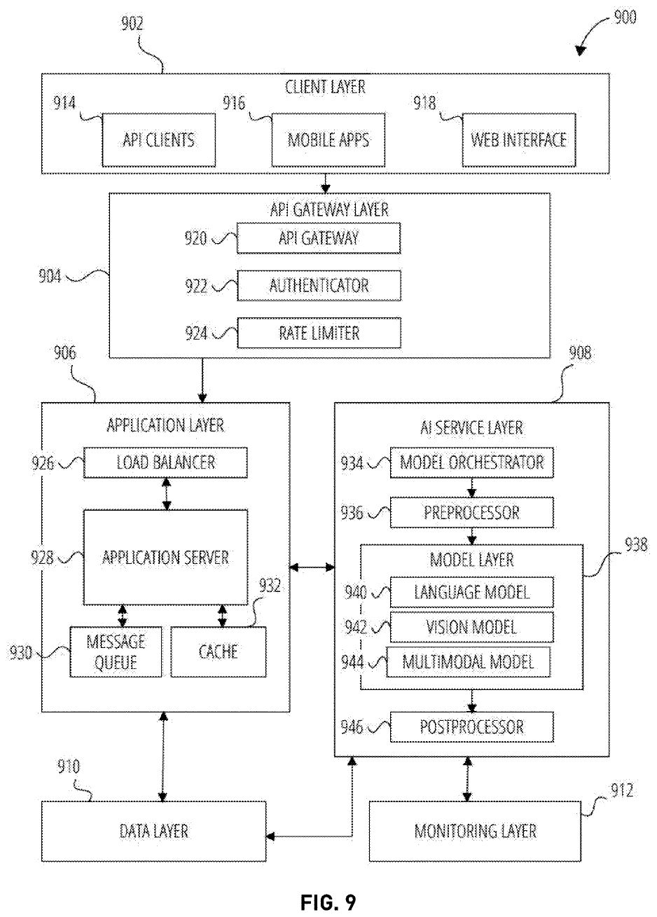

FIG. 9 is a system architecture diagram showing a layered view of an AI integrated system 900 for providing physical activity guidance, according to some examples.

The AI integrated system 900 comprises six example layers: a client layer 902, an API gateway layer 904, an application layer 906, an AI service layer 908, a data layer 910, and a monitoring layer 912.

The client layer 902 provides multiple access points for system interaction related to physical activity guidance. It includes API clients 914, which may enable programmatic access to the capabilities of the AI integrated system 900 and integration with third-party fitness systems and services. Mobile applications 916 facilitate access from mobile devices serving as external body trackers, capturing the user's movements during physical activities like yoga. These mobile applications implement native protocols while maintaining consistent communication patterns with backend systems. A web interface 918 enables browser-based access to the system's capabilities for activity configuration and session review, communicating with lower layers using standard Hypertext Transfer Protocol (HTTP)/Hypertext Transfer Protocol Secure (HTTPS) protocols.

The API gateway layer 904 serves as an entry point for client communications, particularly for transmitting movement data from mobile devices. It contains an API gateway 920, which may implement routing logic to direct incoming body tracking data to appropriate backend services. An authenticator 922 validates user credentials and device connections, for example using industry-standard protocols such as OAuth 2.0 (Open Authorization 2.0) or JWT (JSON Web Tokens). A rate limiter 924 operates to maintain optimal performance during real-time physical activity sessions by preventing system overload.

The application layer 906 encompasses processing capabilities for physical activity data. It features a load balancer 926 that may distribute incoming movement data across multiple application server 928 instances to support resource utilization and system reliability during concurrent sessions. The application server 928 may implement the business logic of physical activity guidance, processing body position data and coordinating responses across various system components. A message queue 930 facilitates asynchronous processing capabilities, enabling buffering of movement data to maintain continuous feedback. A cache 932 operates within this layer to improve response times for frequently accessed reference poses and activity sequences.

The AI service layer 908 provides artificial intelligence capabilities specifically for analyzing physical movements and generating guidance. It includes a model orchestrator 934 that coordinates the execution of various AI models, managing resource allocation for analyzing different aspects of user posture and alignment. A preprocessor 936 prepares input data such as body tracking information and camera images for model consumption, implementing necessary transformations to standardize pose data.

The model layer 938 may include multiple model types tailored for physical activity analysis including for example: language models 940 for generating detailed form corrections and instructional guidance; vision models 942 for analyzing camera images of the user's physical movements; and multimodal models 944 that combine body tracking data with image analysis to provide comprehensive pose evaluation. A postprocessor 946 transforms model outputs into guidance instructions suitable for display on AR glasses, including visual overlays and audio cues.

The data layer 910 provides persistent storage capabilities for physical activity data. While not detailed in the diagram, this layer may include components such as a primary database for maintaining user profiles and session history, a vector database for storing reference poses and alignment patterns, and a file storage component for managing instructional content and session recordings.

The monitoring layer 912 provides observability capabilities across the AI integrated system 900. This may include logging for capturing user session events, metrics collection for analyzing performance of physical activity guidance, and distributed tracing for optimizing feedback latency during real-time sessions.

Communication between layers may follow defined patterns to support real-time feedback. External communications between mobile devices and AR glasses may implement appropriate encryption protocols, for example utilizing Transport Layer Security (TLS)/Secure Sockets Layer (SSL) for data protection. Internal communications may use various protocols selected for specific use cases, for example, Hypertext Transfer Protocol version 2 (HTTP/2), gRPC (Google Remote Procedure Call), or proprietary protocols where appropriate for minimizing latency.

Focusing on the model layer 938, this layer may incorporate various types of machine learning models specialized for physical activity analysis. Neural networks may be deployed across the language, vision, and multimodal models, implementing various architectures suited to specific processing needs. These may include, merely for example, convolutional neural networks (CNNs) for analyzing user images, pose estimation networks for tracking body joint positions, and transformer networks for generating contextual feedback.

The language models 940 may use transformer-based architectures (e.g., Bidirectional Encoder Representations from Transformers (BERT), Generative Pre-trained Transformer (GPT) variants, Text-to-Text Transfer Transformer (T5)) for generating detailed form instructions and personalized guidance based on detected alignment issues. The vision models 942 may employ specialized neural network architectures (e.g., convolutional neural networks (CNNs), Vision Transformers (ViT), Residual Neural Network (ResNet) variants) for analyzing images of the user performing physical activities and identifying specific areas requiring attention.

The multimodal models 944 may implement hybrid architectures designed to process and correlate information across different input modalities, such as skeletal tracking data from the mobile device and visual data from camera feeds. These multimodal models 944 may combine positional, visual, and contextual data to provide integrated analysis that identifies specific alignment issues such as foot placement, hip alignment, spinal extension, and arm engagement.

The AI service layer 908 may also provide pose comparison models for matching the user's current body position against reference poses, measuring deviations in joint angles and body segment orientations. Pose stability analysis models may evaluate the user's balance and form maintenance during held positions. The system may incorporate progression models to track improvements over time and adjust difficulty levels based on user performance.

Each model type may be implemented using various deployment strategies, with the model orchestrator 934 managing their execution and resource allocation to ensure optimal performance and efficient system operation. This includes coordinating the deployment and execution of these various model types, ensuring low-latency feedback during physical activity sessions, and appropriate model selection based on specific pose requirements and user skill levels.

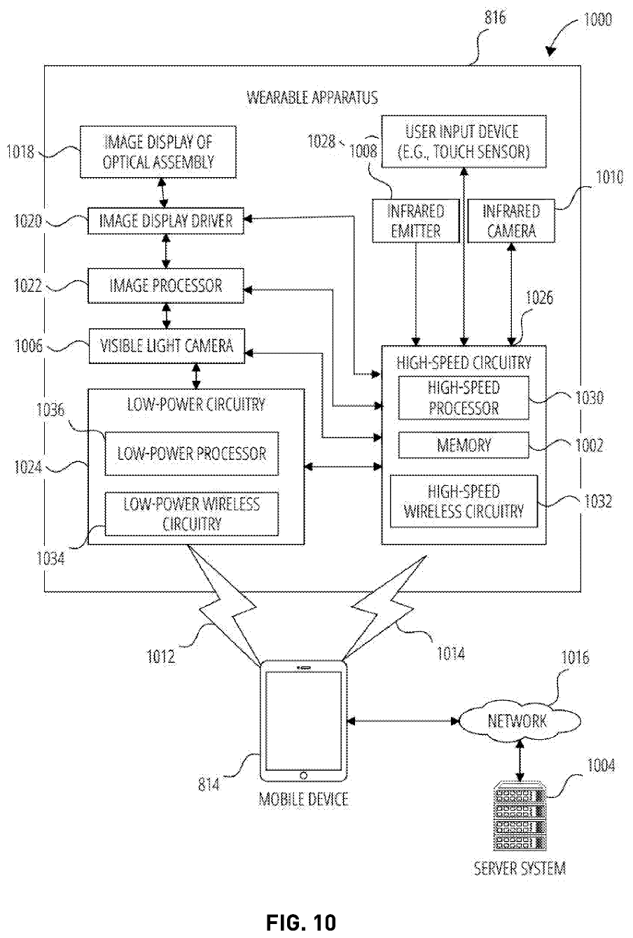

FIG. 10 illustrates a system 1000 including a wearable apparatus 816 with a selector input device, according to some examples. FIG. 10 is a high-level functional block diagram of an example wearable apparatus 816 communicatively coupled to a mobile device 814 and various server systems 1004 (e.g., the server system 810) via various Networks 808.

The wearable apparatus 816 includes one or more cameras, each of which may be, for example, a visible light camera 1006, an infrared emitter 1008, and an infrared camera 1010.

The mobile device 814 connects with wearable apparatus 816 using both a low-power wireless connection 1012 and a high-speed wireless connection 1014. The mobile device 814 is also connected to the server system 1004 and the Network 1016.

The wearable apparatus 816 further includes two image displays of the image display of optical assembly 1018. The two image displays of optical assembly 1018 include one associated with the left lateral side and one associated with the right lateral side of the wearable apparatus 816. The wearable apparatus 816 also includes an image display driver 1020, an image Processor 1022, low-power circuitry 1024, and high-speed circuitry 1026. The image display of optical assembly 1018 is for presenting images and videos, including an image that can include a graphical user interface to a user of the wearable apparatus 816.

The image display driver 1020 commands and controls the image display of optical assembly 1018. The image display driver 1020 may also convert image data into appropriate electrical signals compatible with the display technology used in the image display of optical assembly 1018, potentially including MIPI DSI (Display Serial Interface) protocol signals, LVDS (Low-Voltage Differential Signaling), or other display interface standards. The image display driver 1020 may also convert the image data into a signal or data format suitable for delivery to the image display device. For example, the image data may be video data formatted according to compression formats, such as H.264 (MPEG-4 Part 10), HEVC, Theora, Dirac, RealVideo RV40, VP8, VP9, or the like, and still image data may be formatted according to compression formats such as Portable Network Group (PNG), Joint Photographic Experts Group (JPEG), Tagged Image File Format (TIFF) or exchangeable image file format (EXIF) or the like.

The wearable apparatus 816 includes a frame and stems (or temples) extending from a lateral side of the frame. The wearable apparatus 816 further includes a user input device 1028 (e.g., touch sensor or push button), including an input surface on the wearable apparatus 816. The user input device 1028 (e.g., touch sensor or push button) is to receive from the user an input selection to manipulate the graphical user interface of the presented image.

The components shown in FIG. 10 for the wearable apparatus 816 are located on one or more circuit boards, for example a PCB or flexible PCB, in the rims or temples. Alternatively, or additionally, the depicted components can be located in the chunks, frames, hinges, or bridge of the wearable apparatus 816. Left and right visible light cameras 1006 can include digital camera elements such as a complementary metal oxide-semiconductor (CMOS) image sensor, charge-coupled device, camera lenses, or any other respective visible or light-capturing elements that may be used to capture data, including images of scenes with unknown objects.

The wearable apparatus 816 includes a memory 1002, which stores instructions to perform a subset, or all the functions described herein. The memory 1002 can also include storage device.

As shown in FIG. 10, the high-speed circuitry 1026 includes a high-speed Processor 1030, a memory 1002, and high-speed wireless circuitry 1032. In some examples, the image display driver 1020 is coupled to the high-speed circuitry 1026 and operated by the high-speed Processor 1030 to drive the left and right image displays of the image display of optical assembly 1018. The high-speed Processor 1030 may be any processor capable of managing high-speed communications and operation of any general computing system needed for the wearable apparatus 816. The high-speed Processor 1030 includes processing resources needed for managing high-speed data transfers on a high-speed wireless connection 1014 to a wireless local area network (WLAN) using the high-speed wireless circuitry 1032. In certain examples, the high-speed Processor 1030 executes an operating system such as a LINUX operating system or other such operating system of the wearable apparatus 816, and the operating system is stored in the memory 1002 for execution. In addition to any other responsibilities, the high-speed Processor 1030 executing a software architecture for the wearable apparatus 816 is used to manage data transfers with high-speed wireless circuitry 1032. In certain examples, the high-speed wireless circuitry 1032 is configured to implement Institute of Electrical and Electronic Engineers (IEEE) 802.11 communication standards, also referred to herein as WI-FI®. In some examples, other high-speed communications standards may be implemented by the high-speed wireless circuitry 1032.

The low-power wireless circuitry 1034 and the high-speed wireless circuitry 1032 of the wearable apparatus 816 can include short-range transceivers (e.g., Bluetooth™, Bluetooth LE, Zigbee, ANT+) and wireless wide, local, or wide area Network transceivers (e.g., cellular or WI-FI®). Mobile device 814, including the transceivers communicating via the low-power wireless connection 1012 and the high-speed wireless connection 1014, may be implemented using details of the architecture of the wearable apparatus 816, as can other elements of the Network 1016.

The memory 1002 includes any storage device capable of storing various data and applications, including, among other things, camera data generated by the left and right visible light cameras 1006, the infrared camera 1010, and the image Processor 1022, as well as images generated for display by the image display driver 1020 on the image displays of the image display of optical assembly 1018. While the memory 1002 is shown as integrated with high-speed circuitry 1026, in some examples, the memory 1002 may be an independent standalone element of the wearable apparatus 816. In certain such examples, electrical routing lines may provide a connection through a chip that includes the high-speed Processor 1030 from the image Processor 1022 or the low-power Processor 1036 to the memory 1002. In some examples, the high-speed Processor 1030 may manage addressing of the memory 1002 such that the low-power Processor 1036 will boot the high-speed Processor 1030 any time that a read or write operation involving memory 1002 is needed.

As shown in FIG. 10, the low-power Processor 1036 or high-speed Processor 1030 of the wearable apparatus 816 can be coupled to the camera (visible light camera 1006, infrared emitter 1008, or infrared camera 1010), the image display driver 1020, the user input device 1028 (e.g., touch sensor or push button), and the memory 1002.

The wearable apparatus 816 is connected to a host computer. For example, the wearable apparatus 816 is paired with the mobile device 814 via the high-speed wireless connection 1014 or connected to the server system 1004 via the Network 1016. The server system 1004 may be one or more computing devices as part of a service or network computing system, for example, that includes a processor, a memory, and network communication interface to communicate over the Network 1016 with the mobile device 814 and the wearable apparatus 816.

The mobile device 814 includes a processor and a Network communication interface coupled to the processor. The Network communication interface allows for communication over the Network 1016, low-power wireless connection 1012, or high-speed wireless connection 1014. Mobile device 814 can further store at least portions of the instructions in the memory of the mobile device 814 memory to implement the functionality described herein.

Output components of the wearable apparatus 816 include visual components, such as a display such as a liquid crystal display (LCD), a plasma display panel (PDP), a light-emitting diode (LED) display, a projector, or a waveguide. The image displays of the optical assembly are driven by the image display driver 1020. The output components of the wearable apparatus 816 further include acoustic components (e.g., speakers), haptic components (e.g., a vibratory motor), other signal generators, and so forth. The input components of the wearable apparatus 816, the mobile device 814, and server system 1004, such as the user input device 1028, may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instruments), tactile input components (e.g., a physical button, a touch screen that provides location and force of touches or touch gestures, or other tactile input components), audio input components (e.g., a microphone), and the like.

The wearable apparatus 816 may also include additional peripheral device elements. Such peripheral device elements may include sensors and display elements integrated with the wearable apparatus 816. For example, peripheral device elements may include any I/O components including output components, motion components, position components, or any other such elements described herein.

In some examples, the wearable apparatus 816 may include biometric components or sensors s to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The biometric components may include a brain-machine interface (BMI) system that allows communication between the brain and an external device or machine. This may be achieved by recording brain activity data, translating this data into a format that can be understood by a computer, and then using the resulting signals to control the device or machine.

Example types of BMI technologies, including:
1. Electroencephalography (EEG) based BMIs, which record electrical activity in the brain using electrodes placed on the scalp.
2. Invasive BMIs, which used electrodes that are surgically implanted into the brain.
3. Optogenetics BMIs, which use light to control the activity of specific nerve cells in the brain.

Any biometric data collected by the biometric components is captured and stored with only user approval and deleted on user request, and in accordance with applicable laws. Further, such biometric data may be used for very limited purposes, such as identification verification. To ensure limited and authorized use of biometric information and other personally identifiable information (PII), access to this data is restricted to authorized personnel only, if at all. Any use of biometric data may strictly be limited to identification verification purposes, and the biometric data is not shared or sold to any third party without the explicit consent of the user. In addition, appropriate technical and organizational measures are implemented to ensure the security and confidentiality of this sensitive information.

The motion components include acceleration sensor components (e.g., accelerometer), gravitation sensor components, rotation sensor components (e.g., gyroscope), and so forth. The position components include location sensor components to generate location coordinates (e.g., a Global Positioning System (GPS) receiver component), Wi-Fi or Bluetooth™ transceivers to generate positioning system coordinates, altitude sensor components (e.g., altimeters or barometers that detect air pressure from which altitude may be derived), orientation sensor components (e.g., magnetometers), and the like. Such positioning system coordinates can also be received over low-power wireless connections 1012 and high-speed wireless connection 1014 from the mobile device 814 via the low-power wireless circuitry 1034 or high-speed wireless circuitry 1032.

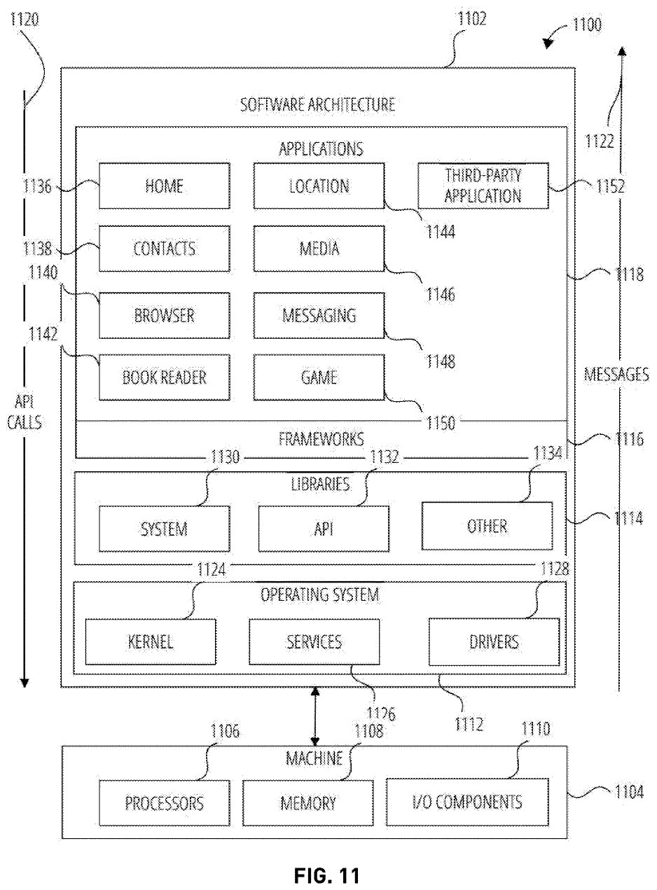

FIG. 11 is a block diagram 1100 illustrating a software architecture 1102, which can be installed on any one or more of the devices described herein. The software architecture 1102 is supported by hardware such as a machine 1104 that includes Processors 1106, memory 1108, and I/O components 1110. In this example, the software architecture 1102 can be conceptualized as a stack of layers, where each layer provides a particular functionality. The software architecture 1102 includes layers such as an operating system 1112, libraries 1114, frameworks 1116, and applications 1118. Operationally, the applications 1118 invoke API calls 1120 through the software stack and receive messages 1122 in response to the API calls 1120.

The operating system 1112 manages hardware resources and provides common services. The operating system 1112 includes, for example, a kernel 1124, services 1126, and drivers 1128. The kernel 1124 acts as an abstraction layer between the hardware and the other software layers. For example, the kernel 1124 provides memory management, processor management (e.g., scheduling), component management, networking, and security settings, among other functionalities. The services 1126 can provide other common services for the other software layers. The drivers 1128 are responsible for controlling or interfacing with the underlying hardware. For instance, the drivers 1128 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 1114 provide a common low-level infrastructure used by the applications 1118. The libraries 1114 can include system libraries 1130 (e.g., C standard library) that provide functions such as memory allocation functions, string manipulation functions, mathematical functions, and the like. In addition, the libraries 1114 can include API libraries 1132 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 1114 can also include a wide variety of other libraries 1134 to provide many other APIs to the applications 1118.

The frameworks 1116 provide a common high-level infrastructure that is used by the applications 1118. For example, the frameworks 1116 provide various graphical user interface (GUI) functions, high-level resource management, and high-level location services. The frameworks 1116 can provide a broad spectrum of other APIs that can be used by the applications 1118, some of which may be specific to a particular operating system or platform.

In an example, the applications 1118 may include a home application 1136, a contacts application 1138, a browser application 1140, a book reader application 1142, a location application 1144, a media application 1146, a messaging application 1148, a game application 1150, and a broad assortment of other applications such as a third-party application 1152. The applications 1118 are programs that execute functions defined in the programs. Various programming languages can be employed to create one or more of the applications 1118, structured in a variety of manners, such as object-oriented programming languages (e.g., Objective-C, Java, or C++) or procedural programming languages (e.g., C or assembly language). In a specific example, the third-party application 1152 (e.g., an application developed using the ANDROID™ or IOS™ software development kit (SDK) by an entity other than the vendor of a platform) may be mobile software running on a mobile operating system such as IOS™, ANDROID™, WINDOWS® Phone, or another mobile operating system. In this example, the third-party application 1152 can invoke the API calls 1120 provided by the operating system 1112 to facilitate functionalities described herein.

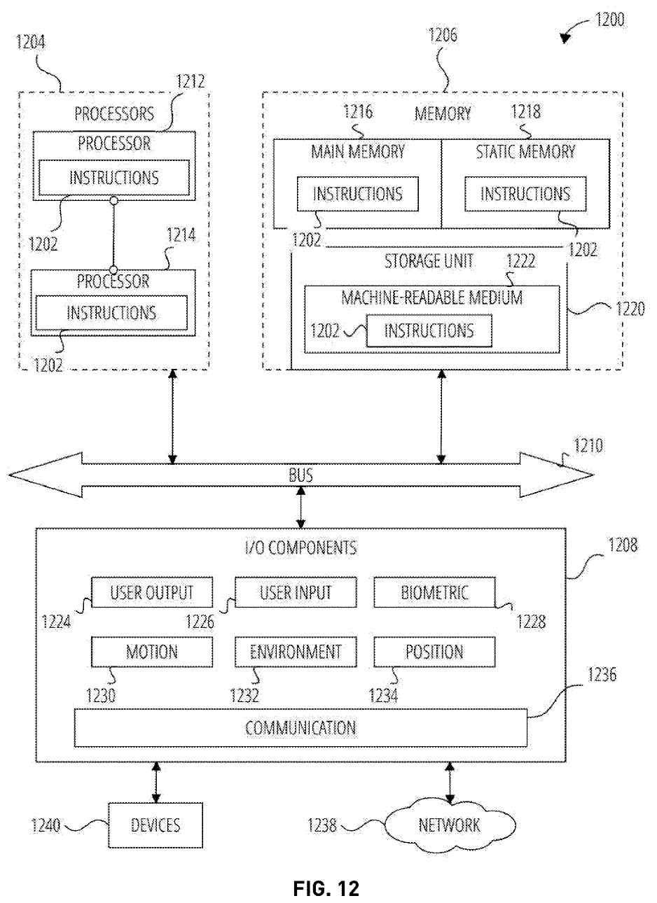

FIG. 12 is a diagrammatic representation of the machine 1200 within which instructions 1202 (e.g., software, a program, an application, an applet, an app, or other executable code) for causing the machine 1200 to perform any one or more of the methodologies discussed herein may be executed. For example, the instructions 1202 may cause the machine 1200 to execute any one or more of the methods described herein. The instructions 1202 transform the general, non-programmed machine 1200 into a particular machine 1200 programmed to carry out the described and illustrated functions in the manner described. The machine 1200 may operate as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 1202, sequentially or otherwise, that specify actions to be taken by the machine 1200. Further, while a single machine 1200 is illustrated, the term “machine” shall also be taken to include a collection of machines that individually or jointly execute the instructions 1202 to perform any one or more of the methodologies discussed herein. The machine 1200, for example, may comprise the user system 802 or any one of multiple server devices forming part of the server system 810. In some examples, the machine 1200 may also comprise both client and server systems, with certain operations of a particular method or algorithm being performed on the server-side and with certain operations of the method or algorithm being performed on the client-side.

The machine 1200 may include Processors 1204, memory 1206, and input/output I/O components 1208, which may be configured to communicate with each other via a bus 1210.

The memory 1206 includes a main memory 1216, a static memory 1218, and a storage unit 1220, both accessible to the Processors 1204 via the bus 1210. The main memory 1206, the static memory 1218, and storage unit 1220 store the instructions 1202 embodying any one or more of the methodologies or functions described herein. The instructions 1202 may also reside, completely or partially, within the main memory 1216, within the static memory 1218, within machine-readable medium 1222 within the storage unit 1220, within at least one of the Processors 1204 (e.g., within the processor's cache memory), or any suitable combination thereof, during execution thereof by the machine 1200.

The I/O components 1208 may include a wide variety of components to receive input, provide output, produce output, transmit information, exchange information, capture measurements, and so on. The specific I/O components 1208 that are included in a particular machine will depend on the type of machine. For example, portable machines such as mobile phones may include a touch input device or other such input mechanisms, while a headless server machine will likely not include such a touch input device. It will be appreciated that the I/O components 1208 may include many other components that are not shown in FIG. 12. In various examples, the I/O components 1208 may include user output components 1224 and user input components 1226. The user output components 1224 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 1226 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 1208 may include biometric components 1228, motion components 1230, environmental components 1232, or position components 1234, among a wide array of other components. For example, the biometric components 1228 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The biometric components may include a brain-machine interface (BMI) system that allows communication between the brain and an external device or machine. This may be achieved by recording brain activity data, translating this data into a format that can be understood by a computer, and then using the resulting signals to control the device or machine.

Example types of BMI technologies, including:
1. Electroencephalography (EEG) based BMIs, which record electrical activity in the brain using electrodes placed on the scalp.
2. Invasive BMIs, which used electrodes that are surgically implanted into the brain.
3. Optogenetics BMIs, which use light to control the activity of specific nerve cells in the brain.

Any biometric data collected by the biometric components is captured and stored only with user approval and deleted on user request, and in accordance with applicable laws. Further, such biometric data may be used for very limited purposes, such as identification verification. To ensure limited and authorized use of biometric information and other personally identifiable information (PII), access to this data is restricted to authorized personnel only, if at all. Any use of biometric data may strictly be limited to identification verification purposes, and the data is not shared or sold to any third party without the explicit consent of the user. In addition, appropriate technical and organizational measures are implemented to ensure the security and confidentiality of this sensitive information.

The motion components 1230 include acceleration sensor components (e.g., accelerometer), gravitation sensor components, rotation sensor components (e.g., gyroscope).

The environmental components 1232 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

With respect to cameras, the user system 802 may have a camera system comprising, for example, front cameras on a front surface of the user system 802 and rear cameras on a rear surface of the user system 802. The front cameras may, for example, be used to capture still images and video of a user of the user system 802 (e.g., “selfies”), which may then be modified with digital effect data (e.g., filters) described above. The rear cameras may, for example, be used to capture still images and videos in a more traditional camera mode, with these images similarly being modified with digital effect data. In addition to front and rear cameras, the user system 802 may also include a 360° camera for capturing 360° photographs and videos.

Moreover, the camera system of the user system 802 may be equipped with multi-camera arrays that may include sensors with different pixel densities, apertures, and focal lengths. Depth-sensing cameras may employ structured light, time-of-flight, or stereo-vision techniques to generate depth maps with precision typically ranging from millimeters to centimeters depending on the distance from the subject. These multiple-camera arrays may include a wide camera, an ultra-wide camera, a telephoto camera, a macro camera, and a depth sensor, for example.

Communication may be implemented using a wide variety of technologies. The I/O components 1208 further include communication components 1236 operable to couple the machine 1200 to a Network 1238 or devices 1240 via respective coupling or connections. For example, the communication components 1236 may include a network interface component or another suitable device to interface with the Network 1238. In further examples, the communication components 1236 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1240 may be another machine or any of a wide variety of peripheral devices (e.g., a peripheral device coupled via a USB).

Moreover, the communication components 1236 may detect identifiers or include components operable to detect identifiers. For example, the communication components 1236 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph™, MaxiCode, PDF417, Ultra Code, UCC RSS-2D bar code, and other optical codes), or acoustic detection components (e.g., microphones to identify tagged audio signals). In addition, a variety of information may be derived via the communication components 1236, such as location via Internet Protocol (IP) geolocation, location via Wi-Fi® signal triangulation, location via detecting an NFC beacon signal that may indicate a particular location, and so forth.

The various memories (e.g., main memory 1216, static memory 1218, and memory of the Processors 1204) and storage unit 1220 may store one or more sets of instructions and data structures (e.g., software) embodying or used by any one or more of the methodologies or functions described herein. These instructions (e.g., the instructions 1202), when executed by Processors 1204, cause various operations to implement the disclosed examples.

The instructions 1202 may be transmitted or received over the Network 1238, using a transmission medium, via a network interface device (e.g., a network interface component included in the communication components 1236) and using any one of several well-known transfer protocols (e.g., hypertext transfer protocol (HTTP)). Similarly, the instructions 1202 may be transmitted or received using a transmission medium via a coupling (e.g., a peer-to-peer coupling) to the devices 1240.

### EXAMPLE STATEMENTS

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.

Example 1 is a method for providing physical activity guidance, comprising: receiving, by one or more processors, movement data of a user from a mobile device positioned to capture the user performing a physical activity; analyzing, by the one or more processors, the movement data to determine alignment of user body positions; generating real-time feedback regarding the user's body positions based on the analysis; and causing presentation of spatially-aligned guidance via augmented reality (AR) glasses based on the generated feedback.

In Example 2, the subject matter of Example 1, wherein receiving movement data comprises: capturing images of the user via a camera of the mobile device; and generating body tracking data based on the captured images.

In Example 3, the subject matter of any one or more of Examples 1-2, wherein analyzing the movement data comprises: comparing the user's current body position to reference positions using a large language model; identifying specific areas requiring improvement; and determining when to advance to subsequent positions.

In Example 4, the subject matter of any one or more of Examples 1-3, wherein causing presentation of spatially-aligned guidance comprises: displaying a virtual instructor demonstrating proper form; overlaying visual guides on a representation of the user's body; and providing audio cues for correcting user form.

In Example 5, the subject matter of any one or more of Examples 1-4, further comprising: capturing periodic static pose data during held positions; generating detailed form feedback; and presenting the feedback through the AR glasses.

In Example 6, the subject matter of any one or more of Examples 1-5, further comprising: maintaining user progress data; adjusting difficulty based on performance; and providing session summaries.

In Example 7, the subject matter of any one or more of Examples 1-6, wherein the movement data comprises: body joint positions; body segment orientations; and movement trajectories.

In Example 8, the subject matter of any one or more of Examples 1-7, wherein analyzing comprises: evaluating foot placement; assessing hip alignment; analyzing spinal extension; and checking arm engagement.

In Example 9, the subject matter of any one or more of Examples 1-8, wherein generating feedback comprises: identifying strengths in user form; determining areas for improvement; generating specific correction instructions; and providing encouragement.

In Example 10, the subject matter of any one or more of Examples 1-9, further comprising: detecting when user turns away from virtual instructor; modifying display of virtual instructor; and maintaining guidance visibility.

In Example 11, the subject matter of any one or more of Examples 1-10, further comprising: displaying a camera frustum for mobile device positioning; guiding proper camera placement; and confirming adequate user visibility.

In Example 12, the subject matter of any one or more of Examples 1-11, further comprising: tracking pose hold duration; monitoring form stability; and providing timing feedback.

In Example 13, the subject matter of any one or more of Examples 1-12, wherein the feedback comprises: visual indicators highlighting body areas; textual instructions for adjustments; and audio guidance for improvements.

In Example 14, the subject matter of any one or more of Examples 1-13, further comprising: storing session data; analyzing progress over time; and generating improvement recommendations.

In Example 15, the subject matter of any one or more of Examples 1-14, further comprising: authenticating user; managing session parameters; and synchronizing data across devices.

In Example 16, the subject matter of any one or more of Examples 1-15, wherein presenting guidance comprises: spatially registering virtual content; maintaining proper alignment; and adjusting based on user movement.

Example 17 is one or more processors comprising processing circuitry to perform operations comprising: receiving movement data of a user from a mobile device positioned to capture the user performing a physical activity; analyzing the movement data to determine alignment of user body positions; generating real-time feedback regarding the user's body positions based on the analysis; and causing presentation of spatially-aligned guidance via augmented reality (AR) glasses based on the generated feedback.

Example 18 is a server system for providing physical activity guidance, comprising: an API gateway configured to receive movement data from a mobile device; a model orchestrator configured to: coordinate processing of the received movement data through multiple AI models; generate real-time feedback based on the processed movement data; and transmit the feedback for presentation via AR glasses.

In Example 19, the subject matter of Example 18, wherein the API gateway comprises: an authenticator for validating requests; a rate limiter for managing request flow; and load balancing functionality for distributing requests.

In Example 20, the subject matter of any one or more of Examples 18-19, wherein the multiple AI models comprise at least one: a language model; a vision model; or a multimodal model.

In Example 21, the subject matter of any one or more of Examples 18-20, further comprising: a preprocessor configured to prepare movement data for model processing; a postprocessor configured to format model outputs; and a message queue for managing data flow.

In Example 22, the subject matter of any one or more of Examples 18-21, further comprising: a data layer for storing user profiles and session data; a monitoring layer for tracking system performance; and a cache for temporary data storage.

In Example 23, the subject matter of any one or more of Examples 18-22, wherein the model orchestrator is configured to: analyze body positioning data; identify alignment issues; generate correction instructions; and determine pose advancement timing.

In Example 24, the subject matter of any one or more of Examples 18-23, wherein generating feedback comprises: comparing current poses to reference poses; identifying specific areas requiring improvement; generating detailed form corrections; and providing encouragement.

In Example 25, the subject matter of any one or more of Examples 18-24, further comprising: an application server configured to: manage user sessions; coordinate data flow; handle client communications.

In Example 26, the subject matter of any one or more of Examples 18-25, wherein the API gateway is configured to: receive image data from mobile devices; validate data format and quality; route data to appropriate processing components.

In Example 27, the subject matter of any one or more of Examples 18-26, further comprising: a database server configured to: store user profiles; track session history; maintain pose reference data.

In Example 28, the subject matter of any one or more of Examples 18-27, wherein the model orchestrator is configured to: manage model selection; coordinate parallel processing; optimize response timing.

In Example 29, the subject matter of any one or more of Examples 18-28, further comprising: third-party server interfaces for: external data integration; additional processing capabilities; expanded functionality.

In Example 30, the subject matter of any one or more of Examples 18-29, wherein generating feedback comprises: evaluating foot placement; assessing hip alignment; analyzing spinal extension; checking arm engagement.

In Example 31, the subject matter of any one or more of Examples 18-30, further comprising: web server functionality for: serving web interfaces; handling HTTP requests; managing client sessions.

In Example 32, the subject matter of any one or more of Examples 18-31, wherein the API gateway implements: request authentication; rate limiting; load balancing; request routing.

Example 33 is a computing apparatus comprising: a processor; and a memory storing instructions that, when executed by the processor, configure the apparatus to: receive, by one or more processors, movement data of a user from a mobile device positioned to capture the user performing a physical activity; analyze, by the one or more processors, the movement data to determine alignment of user body positions; generate real-time feedback regarding the user's body positions based on the analysis; and cause presentation of spatially-aligned guidance via augmented reality (AR) glasses based on the generated feedback.

Example 34 is a non-transitory computer-readable storage medium, the computer-readable storage medium including instructions that when executed by a computer, cause the computer to: receive, by one or more processors, movement data of a user from a mobile device positioned to capture the user performing a physical activity; analyze, by the one or more processors, the movement data to determine alignment of user body positions; generate real-time feedback regarding the user's body positions based on the analysis; and cause presentation of spatially-aligned guidance via augmented reality (AR) glasses based on the generated feedback.

Example 35 is a mobile device for providing physical activity guidance, comprising: a camera configured to capture images of a user performing a physical activity; a processor configured to: analyze the captured images using a body tracking model to generate movement data, cause display of a camera frustum visualization to guide camera positioning relative to the user, transmit the movement data and images to augmented reality (AR) glasses, and receive and present feedback via the AR glasses.

In Example 36, the subject matter of Example 35, wherein displaying the camera frustum visualization comprises showing a graphical representation of the camera's field of view, providing visual guidance for achieving proper camera placement, and confirming when the user's full body is visible within the field of view.

In Example 37, the subject matter of any one or more of Examples 35-36, wherein the processor is further configured to detect when the user turns away from a virtual instructor, modify display of guidance elements, and maintain visibility of key instruction components.

In Example 38, the subject matter of any one or more of Examples 35-37, wherein analyzing the captured images comprises identifying body joint positions, determining body segment orientations, and tracking movement trajectories.

In Example 39, the subject matter of any one or more of Examples 35-38, wherein the processor is further configured to capture periodic static pose images during held positions, transmit the static pose images for detailed analysis, and present resulting feedback through the AR glasses.

In Example 40, the subject matter of any one or more of Examples 35-39, further comprising a wireless communication interface for transmitting data to the AR glasses, an application layer for processing captured images, and memory for storing movement data.

In Example 41, the subject matter of any one or more of Examples 35-40, wherein the processor is configured to maintain a connection with the AR glasses, synchronize data transmission timing, and manage communication protocols.

In Example 42, the subject matter of any one or more of Examples 35-41, wherein the camera is configured to adjust capture parameters based on lighting conditions, maintain focus on the user's full body, and optimize frame rate for movement tracking.

In Example 43, the subject matter of any one or more of Examples 35-42, wherein the processor is configured to authenticate user sessions, manage data privacy settings, and secure communications with the AR glasses.

In Example 44, the subject matter of any one or more of Examples 35-43, wherein analyzing the captured images comprises preprocessing image data, applying the body tracking model, and formatting results for transmission.

In Example 45, the subject matter of any one or more of Examples 35-44, wherein the processor is configured to monitor network connectivity, manage data buffering, and handle transmission interruptions.

In Example 46, the subject matter of any one or more of Examples 35-45, wherein displaying guidance comprises showing visual alignment indicators, providing setup instructions, and confirming proper positioning.

In Example 47, the subject matter of any one or more of Examples 35-46, wherein the processor is configured to store session data, track user progress, and generate session summaries.

In Example 48, the subject matter of any one or more of Examples 35-47, wherein the processor is configured to coordinate with third-party applications, integrate external data sources, and expand system functionality.

In Example 49, the subject matter of any one or more of Examples 35-48, wherein the processor is configured to manage system resources, optimize performance, and handle background processes.

In Example 50, the subject matter of any one or more of Examples 35-49, wherein the processor is configured to implement error handling, provide status updates, and maintain system stability.

### TERM EXAMPLES

“Carrier signal” may include, for example, any intangible medium that can store, encoding, or carrying instructions for execution by the machine and includes digital or analog communications signals or other intangible media to facilitate communication of such instructions. Instructions may be transmitted or received over a network using a transmission medium via a network interface device.

“Client device” may include, for example, any machine that interfaces to a network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

“Component” may include, for example, a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application-specific integrated circuit (ASIC). A hardware component may also include programmable logic or circuitry that is temporarily configured by software to perform certain operations. For example, a hardware component may include software executed by a general-purpose processor or other programmable processors. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” may refer to a hardware component implemented using one or more processors. Similarly, the methods described herein may be at least partially Processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

“Computer-readable storage medium” may include, for example, both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine storage medium” may include, for example, a single or multiple storage devices and media (e.g., a centralized or distributed database, and associated caches and servers) that store executable instructions, routines, and data. The term shall accordingly be taken to include, but not be limited to, solid-state memories, and optical and magnetic media, including memory internal or external to processors. Specific examples of machine-storage media, computer-storage media, and device-storage media include non-volatile memory, including by way of example semiconductor memory devices, e.g., erasable programmable read-only memory (EPROM), electrically erasable programmable read-only memory (EEPROM), Field-Programmable Gate Arrays (FPGA), flash memory devices, Solid State Drives (SSD), and Non-Volatile Memory Express (NVMe) devices; magnetic disks such as internal hard disks and removable disks; magneto-optical disks; and CD-ROM, DVD-ROM, Blu-ray Discs, and Ultra HD Blu-ray discs. In addition, machine storage medium may also refer to cloud storage services, Network Attached Storage (NAS), Storage Area Networks (SAN), and object storage devices. The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Network” may include, for example, one or more portions of a network that may be an ad hoc network, an intranet, an extranet, a Virtual Private Network (VPN), a Local Area Network (LAN), a Wireless LAN (WLAN), a Wide Area Network (WAN), a Wireless WAN (WWAN), a Metropolitan Area Network (MAN), the Internet, a portion of the Internet, a portion of the Public Switched Telephone Network (PSTN), a Voice over IP (VoIP) network, a cellular telephone network, a 5G™ network, a wireless network, a Wi-Fi® network, a Wi-Fi 6® network, a Li-Fi network, a Zigbee® network, a Bluetooth® network, another type of network, or a combination of two or more such networks. For example, a network or a portion of a network may include a wireless or cellular network, and the coupling may be a Code Division Multiple Access (CDMA) connection, a Global System for Mobile communications (GSM) connection, or other types of cellular or wireless coupling. In this example, the coupling may implement any of a variety of types of data transfer technology, such as third Generation Partnership Project (3GPP) including 4G, fifth-generation wireless (5G) networks, Universal Mobile Telecommunications System (UMTS), High Speed Packet Access (HSPA), Long Term Evolution (LTE) standard, others defined by various standard-setting organizations, other long-range protocols, or other data transfer technology.

“Non-transitory computer-readable storage medium” may include, for example, a tangible medium that is capable of storing, encoding, or carrying the instructions for execution by a machine.

“Processor” may include, for example, data processors such as a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), a Quantum Processing Unit (QPU), a Tensor Processing Unit (TPU), a Neural Processing Unit (NPU), a Field Programmable Gate Array (FPGA), another processor, or any suitable combination thereof. The term “processor” may include multi-core processors that may comprise two or more independent processors (sometimes referred to as “cores”) that may execute instructions contemporaneously. These cores can be homogeneous (e.g., all cores are identical, as in multicore CPUs) or heterogeneous (e.g., cores are not identical, as in many modern GPUs and some CPUs). In addition, the term “processor” may also encompass systems with a distributed architecture, where multiple processors are interconnected to perform tasks in a coordinated manner. This includes cluster computing, grid computing, and cloud computing infrastructures. Furthermore, the processor may be embedded in a device to control specific functions of that device, such as in an embedded system, or it may be part of a larger system, such as a server in a data center. The processor may also be virtualized in a software-defined infrastructure, where the processor's functions are emulated in software.

“Signal medium” may include, for example, an intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

“User device” may include, for example, a device accessed, controlled or owned by a user and with which the user interacts perform an action, engagement or interaction on the user device, including an interaction with other users or computer systems.

Augmented Reality (AR)—A technology that overlays digital information, such as images, text, or graphics, onto the physical world, enhancing the user's perception of their environment.

Large Language Model (LLM)—A type of artificial intelligence model designed to process and generate human-like text based on input prompts. In the context of this invention, the LLM analyzes movement data and provides real-time feedback.

Body Tracking—The process of capturing and analyzing the positions and movements of a user's body using computer vision techniques, typically through a camera or sensor.

Camera Frustum—A graphical representation of the field of view of a camera, used to guide users in positioning the camera to ensure optimal visibility and tracking accuracy.

Alignment Guides—Visual overlays presented through AR glasses that assist users in maintaining proper posture and positioning during physical activities.

Virtual Instructor—A digital representation of a trainer or guide displayed through AR glasses, demonstrating proper form and providing visual cues for physical activities.

Pose Advancement—The process of determining when a user should transition from one physical position or exercise to the next, based on their performance and alignment.

Skeletal Representation—A visual model of the user's body, typically displayed as a simplified structure of joints and segments, used for tracking and analyzing movements.

Real-Time Feedback—Immediate guidance provided to the user during physical activities, including visual, textual, and audio cues to improve form and alignment.

Focus Areas—Specific body positions, such as joints or limbs, highlighted by visual indicators to emphasize proper alignment and engagement.

Mobile Tracker—A mobile device, such as a smartphone, used to capture the user's movements via its camera and transmit data for analysis.

Spatially-Aligned Guidance—Feedback presented through AR glasses that is aligned with the user's perspective, ensuring that visual cues remain relevant and visible during physical activities.

Session Data—Information recorded during a physical activity session, including user movements, pose durations, and feedback, used for tracking progress and generating insights.

Distributed Architecture—A system design where computational tasks are divided across multiple components, such as mobile devices, servers, and AR glasses, to enable efficient processing and real-time feedback.

API Gateway—A server component that manages data flow between devices and processing systems, handling tasks such as authentication, rate limiting, and request routing.

Model Orchestrator—A system component that coordinates the processing of movement data through multiple AI models, ensuring efficient analysis and feedback generation.

Visual Indicators—Graphical elements, such as arrows or circular markers, displayed through AR glasses to guide users in refining their posture and alignment.

Pose Hold Duration—The amount of time a user maintains a specific physical position during an activity, tracked by the system to evaluate stability and form.

Spinal Extension—The alignment and positioning of the user's spine during physical activities, analyzed to ensure proper posture and prevent overextension.

Core Muscle Engagement—The activation of central muscle groups, such as the abdominal and back muscles, to stabilize the user's posture and improve overall form.

Adaptive Display—A feature of the AR glasses that adjusts the presentation of guidance elements based on the user's movements or orientation, ensuring continuous visibility.

Textual Feedback—Written instructions provided to the user to refine their form and improve their performance during physical activities.

Audio Cues—Verbal instructions or corrections delivered to the user through AR glasses to enhance their understanding of required adjustments.

Progress Tracking—The process of recording and analyzing user performance over time, enabling the system to provide insights and adjust difficulty levels.

Yoga Lens—A specific implementation of the invention designed for yoga practice, utilizing AR glasses and mobile tracking to provide real-time guidance and feedback.

Avalon—The code name for the prototype AR glasses used in this invention, which integrate augmented reality technology with physical activity guidance features.

Encyclopedia Feature—A system component that provides users with educational content related to physical activities, such as detailed explanations of poses and techniques.

Movement Trajectories—The paths or patterns of motion made by the user's body during physical activities, analyzed to assess alignment and form.

Hands-Free Guidance—A feature of the system that allows users to receive feedback and instructions without needing to interact with external screens or devices.

Session Summary—A post-activity report provided to the user, summarizing their performance, highlighting strengths and areas for improvement, and offering recommendations for future sessions.

Third-Party Integration—The ability of the system to connect with external servers or applications to expand functionality and provide additional capabilities.

Meditation and Breathwork Guidance—An implementation of the invention that uses AR glasses to provide visual and audio cues for breathing exercises and meditation practices.

Group Fitness Applications—A use case where the system enables synchronized physical activity sessions for multiple users, supported by network integration and real-time data sharing.

Workplace Wellness Applications—An implementation of the invention designed to guide users in maintaining proper posture and performing simple exercises during desk work.

Dance and Movement Education—A use case where the system tracks complex dance movements and provides visual feedback to improve technique and timing.

Physical Therapy and Rehabilitation—An implementation of the invention that provides immersive guidance for therapeutic exercises, enabling real-time analysis and progress tracking.

Athletic Training Applications—A use case where the system analyzes complex movement patterns during sports activities to provide detailed feedback and track progress.

Calibrated Setup—The initial phase of system operation where the user adjusts their position and posture to align with the system's requirements for accurate tracking.

Camera Placement Flow—The process of positioning the mobile device to ensure optimal visibility of the user's body for accurate tracking and analysis.

Post-Session Feedback—Insights and recommendations provided to the user after completing a physical activity session, designed to help refine technique and improve future performance.

As used in this disclosure, phrases of the form “at least one of an A, a B, or a C,” “at least one of A, B, or C,” “at least one of A, B, and C,” and the like, should be interpreted to select at least one from the group that comprises “A, B, and C.” Unless explicitly stated otherwise in connection with a particular instance in this disclosure, this manner of phrasing does not mean “at least one of A, at least one of B, and at least one of C.” As used in this disclosure, the example “at least one of an A, a B, or a C,” would cover any of the following selections: {A}, {B}, {C}, {A, B}, {A, C}, {B, C}, and {A, B, C}.

Unless the context clearly requires otherwise, throughout the description and the claims, the words “comprise,” “comprising,” and the like are to be construed in an inclusive sense, as opposed to an exclusive or exhaustive sense, e.g., in the sense of “including, but not limited to.”

As used herein, the terms “connected,” “coupled,” or any variant thereof means any connection or coupling, either direct or indirect, between two or more elements; the coupling or connection between the elements can be physical, logical, or a combination thereof.

Additionally, the words “herein,” “above,” “below,” and words of similar import, when used in this application, refer to this application as a whole and not to any portions of this application. Where the context permits, words using the singular or plural number may also include the plural or singular number respectively.

The word “or” in reference to a list of two or more items, covers all the following interpretations of the word: any one of the items in the list, all the items in the list, and any combination of the items in the list. Likewise, the term “and/or” in reference to a list of two or more items, covers all the following interpretations of the word: any one of the items in the list, all the items in the list, and any combination of the items in the list.

The various features, operations, or processes described herein may be used independently of one another, or may be combined in various ways. All possible combinations and sub-combinations are intended to fall within the scope of this disclosure. In addition, certain method or process blocks may be omitted in some implementations.

Although some examples, e.g., those depicted in the drawings, include a particular sequence of operations, the sequence may be altered without departing from the scope of the present disclosure. For example, some of the operations depicted may be performed in parallel or in a different sequence that does not materially affect the functions as described in the examples. In other examples, different components of an example device or system that implements an example method may perform functions at substantially the same time or in a specific sequence.

What is claimed is:

1. A method for providing physical activity guidance, comprising:
receiving, by one or more processors, movement data of a user from a mobile device positioned to capture the user performing a physical activity;
analyzing, by the one or more processors, the movement data to determine an alignment of body positions of the user;
generating real-time feedback regarding the body positions of the user based on the alignment of the body positions of the user; and
causing presentation of spatially-aligned guidance via augmented reality (AR) glasses based on the generated feedback.

2. The method of claim 1, wherein receiving movement data comprises:
capturing images of the user via a camera of the mobile device; and
generating body tracking data based on the captured images.

3. The method of claim 1, wherein analyzing the movement data comprises:
comparing a current body position of the user to reference positions using a large language model;
identifying specific areas requiring improvement; and
determining when to advance to subsequent positions.

4. The method of claim 1, wherein causing presentation of spatially-aligned guidance comprises:
displaying a virtual instructor demonstrating proper form;
overlaying visual guides on a representation of a body of the user; and
providing audio cues for correcting user form.

5. The method of claim 1, further comprising:
capturing periodic static pose data during held positions;
generating detailed form feedback; and
presenting the feedback through the AR glasses.

6. The method of claim 1, further comprising:
maintaining user progress data;
adjusting difficulty based on performance; and
providing session summaries.

7. The method of claim 1, wherein the movement data comprises:
body joint positions;
body segment orientations; and
movement trajectories.

8. The method of claim 1, wherein analyzing comprises:
evaluating foot placement;
assessing hip alignment;
analyzing spinal extension; and
checking arm engagement.

9. The method of claim 1, wherein generating feedback comprises:
identifying strengths in user form;
determining areas for improvement;
generating specific correction instructions; and
providing encouragement.

10. The method of claim 1, further comprising:
detecting when user turns away from virtual instructor;
modifying display of virtual instructor; and
maintaining guidance visibility.

11. The method of claim 1, further comprising:
displaying a camera frustum for mobile device positioning;
guiding proper camera placement; and
confirming adequate user visibility.

12. The method of claim 1, further comprising:
tracking pose hold duration;
monitoring form stability; and
providing timing feedback.

13. The method of claim 1, wherein the feedback comprises:
visual indicators highlighting body areas;
textual instructions for adjustments; and
audio guidance for improvements.

14. The method of claim 1, further comprising:
storing session data;
analyzing progress over time; and
generating improvement recommendations.

15. The method of claim 1, further comprising:
authenticating user;
managing session parameters; and
synchronizing data across devices.

16. The method of claim 1, wherein presenting guidance comprises:
spatially registering virtual content;
maintaining proper alignment; and
adjusting based on user movement.

17. A computing apparatus comprising:
a processor; and
a memory storing instructions that, when executed by the processor, configure the apparatus to:
receive, by one or more processors, movement data of a user from a mobile device positioned to capture the user performing a physical activity;
analyze, by the one or more processors, the movement data to determine an alignment of body positions of the user;
generate real-time feedback regarding the body positions of the user based on the alignment of the body positions of the user; and
cause presentation of spatially-aligned guidance via augmented reality (AR) glasses based on the generated feedback.

18. The computing apparatus of claim 17, wherein to receive movement data, the instructions configure the apparatus to:
capture images of the user via a camera of the mobile device; and
generate body tracking data based on the captured images.

19. The computing apparatus of claim 17, wherein to analyze the movement data, the instructions configure the apparatus to:
compare a current body position of the user to reference positions using a large language model;
identify specific areas requiring improvement; and
determine when to advance to subsequent positions.

20. A non-transitory computer-readable storage medium, the computer-readable storage medium including instructions that when executed by a computer, cause the computer to:
receive, by one or more processors, movement data of a user from a mobile device positioned to capture the user performing a physical activity;
analyze, by the one or more processors, the movement data to determine an alignment of body positions of the user;
generate real-time feedback regarding the body positions of the user based on the alignment of the body positions of the user; and
cause presentation of spatially-aligned guidance via augmented reality (AR) glasses based on the generated feedback.
